(19) United States
(12) Patent Application Publication
(10) Pub. No.: US 2015/0011427 A1
(43) Pub. Date: Jan. 8, 2015

(54) EVAPORATIVE EDGE LITHOGRAPHY OF A MICROARRAY

(71) Applicant: FLORIDA STATE UNIVERSITY RESEARCH FOUNDATION, TALLAHASSEE, FL (US)

(72) Inventors: STEVEN LENHERT, TALLAHASSEE, FL (US); NICHOLAS VAFAI, TALLAHASSEE, FL (US)

(21) Appl. No.: 14/322,040

(22) Filed: Jul. 2, 2014

Related U.S. Application Data
(63) Continuation-in-part of application No. 14/178,325, filed on Feb. 12, 2014.
(60) Provisional application No. 61/841,980, filed on Jul. 2, 2013.

(30) Foreign Application Priority Data
Jul. 12, 2013 (IB) PCT/IB2013/055762

(51) Int. Cl. G01N 33/50; B01J 19/00

(52) U.S. Cl. CPC G01N 33/5029; B01J 19/0046; B01J 2219/00659; B01J 2219/00599; G01N 2500/10
USPC 506/14; 506/27; 506/15

(57) ABSTRACT

Patterned material depositions are formed on a surface of a substrate following filling each of one or more dispersions between each of spaces bounded by a stencil or a pair of patterned barriers on the surface of the substrate. Each dispersion comprises at least one liquid continuous medium and at least one dispersed material. Each patterned material deposition encompasses at least one dispersed material and is formed by evaporating the liquid continuous medium from the dispersion. Patterned material depositions may have multilayer structures. Systems include methods and device.

### CROSS-REFERENCE TO RELATED APPLICATIONS

This application claims benefit of priority to U.S. Provisional Patent Application No. 61/841,980, entitled “EVAPORATIVE EDGE LITHOGRAPHY (EEL) OF A LIPOSOMAL DRUG MICROARRAY FOR CELL MIGRATION ASSAYS,” filed Jul. 2, 2013, International Application No. PCT/IB2013/055762, entitled “SCALABLE LIPOSOME MICROARRAY SCREENING” filed Jul. 12, 2013, which in turn claims priority to U.S. Provisional Patent Application No. 61/671,214, entitled “SCALABLE LIPOSOME MICROARRAY SCREENING” filed Jul. 13, 2012, and Continuation-in-part application of U.S. patent application Ser. No. 14/178,325, entitled “EVAPORATIVE EDGE LITHOGRAPHY OF A LIPOSOMAL DRUG MICROARRAY FOR CELL MIGRATION ASSAYS,” filed Feb. 12, 2014. The entire contents and disclosures of these patent applications are incorporated herein by reference in their entirety.

This application also makes reference to U.S. Provisional Patent Application No. 61/383,775, entitled “HIGH THROUGHPUT OPTICAL QUALITY CONTROL OF PHOSPHOLIPID MULTILAYER FABRICATION VIA DIP PEN NANOLITHOGRAPHY (DPN),” filed Sep. 17, 2010. U.S. Provisional Patent Application No. 61/387,764, entitled “NOVEL DEVICE FOR DETECTING AND ANALYZING AQUEOUS SAMPLES,” filed Sep. 21, 2010. U.S. Provisional Patent Application No. 61/387,550, entitled “LIPID MULTILAYER GRATINGS,” filed Sep. 29, 2010. U.S. Provisional Patent Application No. 61/387,556, entitled “LIPID MULTILAYER GRATINGS FOR SEMI-SYNTHETIC QUORUM SENSORS,” filed Sep. 29, 2010. U.S. Provisional Patent Application No. 61/451,619, entitled “IRIDESCENT SURFACES AND APPARATUS FOR REAL TIME MEASUREMENT OF LIQUID AND CELLULAR ADHESION,” filed Mar. 11, 2011. U.S. Provisional Patent Application No. 61/451,635, entitled “METHODS AND APPARATUS FOR LIPID MULTILAYER PATTERNING,” filed Mar. 11, 2011. U.S. Provisional Patent Application No. 61/501,298, entitled “LIPOSOME MICROARRAY SURFACE AND THEIR USE FOR CELL CULTURE SCREENING,” filed Jun. 27, 2011. U.S. patent application Ser. No. 13/234,540, entitled “OPTICAL METHOD FOR MEASURING HEIGHT OF FLUORESCENT PHOSPHOLIPID FEATURES FABRICATED VIA DIP-PEN NANOLITHOGRAPHY,” filed Sep. 11, 2011. U.S. patent application Ser. No. 13/238,498, entitled “INTEGRATED DEVICE FOR ANALYZING AQUEOUS SAMPLES USING LIPID MULTILAYER,” filed Sep. 21, 2011. U.S. patent application Ser. No. 13/248,250, entitled “SEMI-SYNTHETIC QUORUM SENSORS,” filed Sep. 29, 2011. U.S. Provisional Patent Application No. 61/570,490, entitled “LIPID MULTILAYER MICROARRAYS FOR IN VITRO LIPOSOMAL DRUG DELIVERY AND SCREENING,” filed Dec. 14, 2011. U.S. Provisional Patent Application No. 61/577,834, entitled “HIGH THROUGHPUT SCREENING METHOD AND APPARATUS,” filed Dec. 20, 2011. U.S. Provisional Patent Application No. 61/577,910, entitled “NANOSTRUCTURED LIPID MULTILAYER FABRICATION AND DEVICES THEREOF,” filed Dec. 20, 2011. U.S. patent application Ser. No. 13/417,650, entitled “IRIDESCENT SURFACES AND APPARATUS FOR REAL TIME MEASUREMENT OF LIQUID AND CELLULAR ADHESION,” filed Mar. 12, 2012. U.S. patent application Ser. No. 13/417,588, entitled “METHODS AND APPARATUS FOR LIPID MULTILAYER PATTERNING,” filed Mar. 12, 2012. U.S. patent application Ser. No. 13/534,772, entitled “LIPID MULTILAYER MICROARRAYS AND THEIR USE FOR CELL CULTURE SCREENING,” filed Jun. 27, 2012. U.S. Provisional Patent Application No. 61/672,505, entitled “SURFACE SUPPORTED LIPOSOME NANOARRAYS AS BIOMIMETIC SENSORS,” filed Jul. 17, 2012. International Patent Application No. PCT/IB2013/055884 to Lenhert et al., entitled “SURFACE SUPPORTED LIPOSOME NANOARRAYS AS BIOMIMETIC SENSORS,” filed Jul. 17, 2013. U.S. patent application Ser. No. 14/178,325, entitled “EVAPORATIVE EDGE LITHOGRAPHY OF A LIPOSOMAL DRUG MICROARRAY FOR CELL MIGRATION ASSAYS,” filed Feb. 12, 2014. The entire disclosure and contents of these patent applications are incorporated herein by reference.

### BACKGROUND

1. Field of the Invention

The present invention relates to evaporative edge lithography of microarrays.
2. Related Art

Better techniques are needed for forming various types of microarrays.

### SUMMARY

According to a first broad aspect, the present invention provides a product comprising one or more arrays of multilayer structures, wherein the product is formed by a method comprising the following steps: (a) forming one or more respective openings in a stencil, wherein the one or more respective openings have respective peripheral edges, (b) disposing the stencil on a surface of a substrate, (c) filling the one or more respective openings in the stencil on the surface of the substrate with one or more respective dispersions, wherein each of the one or more respective dispersions comprises one or more liquid continuous media and one or more respective dispersed materials, and (d) evaporating the one or more liquid continuous media from each of the respective dispersions in the one or more respective openings in the stencil on the surface of the substrate to thereby form one or more patterned arrays of the one or more respective dispersed materials along the respective peripheral edges of the one or more respective openings in the stencil on the surface of the substrate.

According to a second broad aspect, the present invention provides a method comprising the following steps: (a) forming one or more respective openings in a stencil, wherein the one or more respective openings have respective peripheral edges, (b) disposing the stencil on a surface of a substrate, (c) filling the one or more respective openings in the stencil on the surface of the substrate with one or more respective dispersions, wherein each of the one or more respective dispersions comprises one or more liquid continuous media and one or more respective dispersed materials, and (d) evaporating the one or more liquid continuous media from each of the respective dispersions in the one or more respective openings in the stencil on the surface of the substrate to thereby form one or more patterned arrays of the one or more respective dispersed materials along the respective peripheral edges of the one or more respective openings in the stencil on the surface of the substrate.

According to a third broad aspect, the present invention provides a product comprising one or more arrays of multilayer structures, wherein the product is formed by a method comprising the following steps: (a) disposing one or more pairs of barriers on a surface of a substrate to thereby form one or more respective spaces between the one or more pairs of barriers, wherein the one or more pairs of barriers have one or more respective edges, (b) filling the one or more respective spaces between the one or more pairs of barriers with one or more respective dispersions, wherein each of the respective dispersions comprises one or more liquid continuous media and one or more respective dispersed materials, and (c) evaporating the one or more liquid continuous media from each of the one or more respective dispersions in the one or more respective spaces between the one or more pairs of barriers to thereby form one or more patterned arrays of the one or more respective dispersed materials along the one or more respective edges of the one or more pairs of barriers.

According to a fourth broad aspect, the present invention provides a method comprising the following steps: (a) disposing one or more pairs of barriers on a surface of a substrate to thereby form one or more respective spaces between the one or more pairs of barriers, wherein the one or more pairs of barriers have one or more respective edges, (b) filling the one or more respective spaces between the one or more pairs of barriers with one or more respective dispersions, wherein each of the respective dispersions comprises one or more liquid continuous media and one or more respective dispersed materials, and (c) evaporating the one or more liquid continuous media from each of the one or more respective dispersions in the one or more respective spaces between the one or more pairs of barriers to thereby form one or more patterned arrays of the one or more respective dispersed materials along the one or more respective edges of the one or more pairs of barriers.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration of edge evaporation lithography according to one embodiment of the present invention.

FIG. 2 is a diagram showing supramolecular structures of liposomes and surface-supported lipid nanostructures.

FIG. 3 is a diagram showing supramolecular structures of loaded liposomes and surface-supported loaded lipid nanostructures.

FIG. 4 is a diagram showing a lipid nanoarray delivery system according to one embodiment of the present invention.

FIG. 5 is a schematic illustration of edge evaporation lithography of a liposomal drug microarray for cell migration assays according to one embodiment of the present invention.

FIG. 6 is a schematic illustration of edge evaporation lithography and its use for cell migration assay according to one embodiment of the present invention.

FIG. 7 is a schematic illustration of edge evaporation lithography and its use for cell migration assay according to one embodiment of the present invention.

FIG. 8 is a perspective view of a surface of a stencil used for edge evaporation lithography according to one embodiment of the present invention.

FIG. 9 is a perspective view of a surface of a stencil used for edge evaporation lithography according to one embodiment of the present invention.

FIG. 10 shows the chemical structure of 1,2-dioleoyl-3-trimethylammonium-propane (chloride salt) (DOTAP).

FIG. 11 shows the chemical structure of 1,2-dioleoyl-sn-glycerol-3-phosphoethanolamine-N-(lissamine rhodamine b sulfonyl)(ammonium salt) (DOPE-rhodamine).

FIG. 12 is a fluorescent image of arrays at start lipid concentration of 2 μg/ml (selection in white used for intensity profile).

FIG. 13 is a plot profile graph of the vertically average fluorescence intensity profile across the horizontal cross section outlined in FIG. 10.

FIG. 14 is a graph of size of patterns calibrated from fluorescence intensity of edge and middle of a channel bounded by a pair of PDMS strips according to one embodiment of the present invention.

FIG. 15 is a fluorescent image of arrays at lipid concentration of 200 μg/ml.

FIG. 16 is a fluorescent image of arrays at lipid concentration of 20 μg/ml.

FIG. 17 is a graph of fluorescence intensity of lipid patterns at increasing exposure times (slope is sensitivity).

FIG. 18 is a graph sensitivity as a function of concentration.

FIG. 19 Atomic force microscopy of 200 μg/ml lipid after removal of barriers.

FIG. 20 is a micrograph in phase contrast of adherent HeLa cells in channels created from lipid concentrations of 2 μg/ml according to one embodiment of the present invention.

FIG. 21 is a micrograph in phase contrast of HeLa cells spread out and attached on substrate created from lipid at the concentration of 20 mg/ml.

FIG. 22 is a graph of adherent cell density versus the concentration of lipid solutions used to form films in assay channels (concentrations used in FIGS. 18 and 19 are indicated by asterisks).

FIG. 23 is an image showing initial HeLa cell epithelial sheets before migration according to one embodiment of the present invention.

FIG. 24 is an image showing initial HeLa cell epithelial sheets 24 hours after migration according to one embodiment of the present invention.

FIG. 25 is a graph showing the average distance of migration edge of HeLa cells over 24 hours at lipid solution concentrations of 0, 200 ng/ml, 2 μg/ml, 20 μg/ml, 200 μg/ml, 2 mg/ml, and 20 mg/ml (left to right on graph).

FIG. 26 shows the chemical structure of Taxotere® (docetaxel).

FIG. 27 is a micrograph of a HeLa cell strip (in phase contrast) in contact with a DOTAP only fluorescent lipid film (doped with 1 mol % DOPE-rhodamine), 1 hour after polydimethylsiloxane (PDMS) barriers were removed.

FIG. 28 is a micrograph of the HeLa cell strip of FIG. 27, 24 hours after the polydimethylsiloxane (PDMS) were removed.

FIG. 29 is a micrograph of a HeLa strip incubated with a docetaxel encapsulated fluorescent lipid film, 1 hour after polydimethylsiloxane (PDMS) barriers were removed.

FIG. 30 is a micrograph of the HeLa cell strip of FIG. 27, 24 hours after the polydimethylsiloxane (PDMS) were removed.

FIG. 31 is a graph of HeLa migration rate (μm/hr) as a function of drug treatment from lipid multilayer films.

FIG. 32 is a fluorescent micrograph of cells at time 0 hours after contact with lipid encapsulated docetaxel films.

FIG. 33 is a fluorescent micrograph of cells at 24 hours after contact with lipid encapsulated docetaxel films.

FIG. 34 is a graph of migration rate (in μm/hour) versus drug to lipid ratio (by mass).

FIG. 35 is a set of fluorescence images of hexadecane films formed on three different chemically treated surfaces through edge evaporation lithography (EEL).

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “analyte” refers to the conventional meaning of the term “analyte,” i.e., a substance or chemical constituent of a sample that is being detected or measured in a sample. In one embodiment of the present invention, a sample to be analyzed may be an aqueous sample, but other types of samples may also be analyzed using a device of the present invention.

For purposes of the present invention, the term “array” refers to a one-dimensional or two-dimensional set of microstructures and/or cell cultures. An array may be any shape. For example, an array may be a series of microstructures arranged in a line, such as an array of strips, or an array of squares. An array may be arranged in a square or rectangular grid. There may be sections of the array that are separated from other sections of the array by spaces. An array may have other shapes. For example, an array may be a series of microstructures arranged in a series of concentric circles, in a series of concentric squares, a series of concentric triangles, a series of curves, etc. The spacing between sections of an array or between microstructures in any array may be regular or may be different between particular sections or between particular pairs of microstructures. The microstructure arrays of the present invention may be composed of microstructures having zero-dimensional, one-dimensional or two-dimensional shapes. The microstructures having two-dimensional shapes may have shapes such as squares, rectangles, circles, parallelograms, pentagons, hexagons, irregular shapes, etc. An array may be a set of pairs of microstructures. An array may be a set of microstructures wherein each microstructure of the set is in the shape of an enclosure.

For purposes of the present invention, the term “away” refers to increasing the distance between two aligned objects. For example, a contact controlling positioning device may be used to move: a stamp away from an ink palette, an ink palette away from a stamp, a stamp away from a substrate, a substrate away from a stamp, etc.

For purposes of the present invention, the term “barrier” refers to a structure that is used to control the flow of a dispersion on a substrate. In one embodiment, a barrier may be made of an elastomeric material such as polydimethylsiloxane (PDMS), cellophane, polyurethanes, polyimides, and cross-linked Novolac™ resins (a phenol formaldehyde polymer). In other embodiments the barrier may be made of paraffin-based films, photoresists such as SU-8, or an epoxy. Although in the examples of the present invention described below and shown in the drawings, the barriers are rectangular-box shaped, barriers may be any shape such wedge-shaped, oval-shaped, cylindrical-shaped, tubular-shape, hexagonal, triangular-prism-shaped, pentahedron-shaped, star-shaped, etc. Although in the examples described below, the barriers are arranged in pairs, in some embodiments of the present invention the barriers may be isolated from each other allowing a dispersion to be deposited along a single edge of the barrier to thereby form a multilayer structure at the edge of the barrier when the solvent of the lipid solution evaporates. Also, depending on the shape of a barrier, a lipid solution may deposited around the barrier to form a lipid multilayer structure around the barrier when the solvent of the lipid solution evaporates. For example, if the barrier is a cylinder that is stood on one of its ends on a substrate, such a procedure may be used to form a ring-shaped lipid multilayer structure around the cylinder.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, an oligonucleotide, etc.

For purposes of the present invention, the term “bound” and the term “bounded” refer to two or more lipid multilayer structures that define a region of a substrate between the two or more lipid multilayer structures by forming at least one “boundaries” between the bounded region and an exterior region beyond the bound region. The bound region of the substrate may be filled with cell culture so that the lipid multilayer structures bounding the region also bound the cell culture. The cells may or may not also be present on top of the “boundaries” that bound a region of the substrate. For example, two lipid multilayer structures on two parallel sides of a region may define a region of the two lipid multilayer structures between the two multilayer structures and therefore, the two lipid multilayer structures “bound” the region. The two lipid multilayer structures may also bound a cell culture that fills the region. The fact that a pair of lipid multilayer structures may “bound” a cell culture does not mean that the lipid multilayer structures will prevent the cell culture from migrating across the lipid multilayer structures. For example, in one embodiment of the present invention, a pair of lipid multilayer structures, i.e., “boundaries,” are formed along the edges of a pair of barriers of a stencil on a substrate to thereby “bound” a region on the substrate. When a cell culture is deposited in the opening of the substrate, the enclosure will “bound” the cell culture because the walls of the barriers of the stencil prevent the cells of the cell culture from being deposited beyond edges of the barriers. However, once the barriers of the stencil are removed, the cells of the cell culture may migrate beyond the lipid multilayer structures bounding the region of the substrate.

For purposes of the present invention, the term “boundary” refers to one lipid multilayer structure of a pair or a set of lipid multilayer structures that bound a region of a substrate.

For purposes of the present invention, the term “camera” refers to any type of camera or other device that senses light intensity. Examples of cameras include digital cameras, scanners, charged-coupled devices, CMOS sensors, photomultiplier tubes, analog cameras such as film cameras, etc. A camera may include additional lenses and filters such as the lenses of a microscope apparatus that may be adjusted when the camera is calibrated.

For purposes of the present invention, the term “channel” refers to an elongated recess. An example of a channel is the region on a substrate between two barriers. A channel may be straight, zig-zag, curved, etc.

For purposes of the present invention, the term “contacting surface” refers to a surface of a stamp that contacts a surface onto which a pattern comprising lipid ink is to be printed.

For the purpose of the present invention, the term “curing” refers to the toughening or hardening of a polymer material by cross-linking of polymer chains, brought about by electron beams, heat or chemical additives.

For the purpose of the present invention, the term “curing agent” refers to substances or mixtures of substances added to a polymer composition to promote or control the curing reaction. An agent which does not enter into the reaction is known as a catalytic hardener or catalyst. A reactive curing agent or hardener is generally used in much greater amounts than a catalyst, and actually enters into the reaction. Cross-linking agents are distinguished from catalysts because they react with molecules and are coupled directly into the cured system as a structural member of the polymer.

For purposes of the present invention, the term “detector” refers to any type of device that detects or measures light. A camera is a type of detector.

For purposes of the present invention, the term “dispersed” refers to one or more additives being blended with, mixed with, dispersed in, dissolved in, distributed in, suspended in, scattered in, etc., an organic host liquid.

For purposes of the present invention, the term “dispersed material” refers to a material that is blended with, mixed with, dispersed in, dissolved in, distributed in, suspended in, or scattered in one or more liquid continuous media. Two or more different types of dispersed materials may be mixed and be dispersed together in the same liquid continuous media. The dispersed material may be one or more organic liquids, gels, liquid crystals, solid materials such as: organic solids such as small organic molecules, inorganic compounds, biomolecules, polymer materials, glasses, metals, semiconductor materials, fullerenes, etc. In one embodiment of the present invention, the dispersed material may be an organic liquid such as: an alkane such as hexadecane, etc., an alkene, an alkyne, an aromatic material, a fluid fatty acid such as oleic acid, a phosphatidic acid, etc. In another embodiment of the present invention, the dispersed material may be metallic nanoparticles such as gold, silver, iron, iron oxide. In one embodiment of the present invention, the dispersed material may be semiconductor nanoparticles such as CdSe, ZnS, Si quantum dots. A dispersed material may also be microscopic beads such as latex or glass spheres. A dispersed material may be dispersed particles in any shape such as pyramidal, cubic, spherical, irregularly-shaped, etc. For example, the dispersed particles could be spherical beads of glass, plastic, rubber, etc. irregularly-shaped quantum dots of semiconductor materials, fullerenes of any of a variety of shapes, etc.

For purposes of the present invention, the term “dispersion” refers to a system in which one or more dispersed materials are distributed throughout a liquid continuous medium. A dispersion may be classified in a number of different ways, including how large dispersed materials are in relation to the particles of the continuous media or whether or not precipitation occurs. There are three main types of dispersions: molecular dispersions, colloid, and coarse dispersions. Molecular dispersion is a true solution of a solute in a solvent. The dispersed material (solute) is in form of separate molecules homogeneously distributed throughout the liquid continuous medium (solvent). For example, a type of molecular dispersions is an aqueous solution of salts. Colloids are micro-heterogeneous dispersed systems. Generally, the dispersed materials of colloids cannot be separated from the continuous media under gravity, centrifugal or other forces. Dispersed materials of colloids may be separated from the continuous media by micro-filtration. Milk is an example of a colloid. Coarse dispersions are heterogeneous dispersed systems. They are characterized by relatively fast sedimentation of the dispersed materials caused by gravity or other forces. Dispersed materials of coarse dispersions may be easily separated from the continuous media by filtration. In one embodiment of the present invention, a dispersion may encompass one or more types of liquid continuous media and one or more types of dispersed materials.

For purposes of the present invention, the term “dot” refers to a microstructure that has a zero-dimensional shape.

For purposes of the present invention, the term “drug” refers to a material that may have a biological effect on a cell, including but not limited to small organic molecules, inorganic compounds, polymers such as nucleic acids, peptides, saccharides, or other biologic materials, nanoparticles, etc.

For purposes of the present invention, the term “edge” refers to a line of an intersection of two surfaces, a rim, or a brink. When used with respect to a barrier, the term “edge” refers to where the side of a barrier contacts a substrate. When used with respect to an opening in a stencil, the term “edge” refers to where the periphery of the opening contacts a substrate.

For purposes of the present invention, the term “encapsulated” refers to being confined by a lipid multilayer or partitioned within a lipid multilayer structure.

For purposes of the present invention, the term “enclosure” refers to a lipid multilayer structure that has the shape of a closed curve. A lipid multilayer structure in the shape of an enclosure may be formed by using stencil with openings. The stencil is placed on a substrate and a lipid solution having a solvent, one or more lipids and a drug is deposited in the openings of the stencil. When the solvent is evaporated, a lipid multilayer structure in the shape of an enclosure is formed around the edge of each opening where the opening contacts the substrate. An enclosure may be any shape such as circular, oval, square, rectangular, triangular, pentagonal, rectangular, crescent-shaped, star-shaped, lozenge-shaped, etc.

For purposes of the present invention, the term “fullerene” refers to any molecule composed entirely of carbon, in the form of a hollow sphere, ellipsoid, tube, and many other shapes. Spherical fullerenes are also called buckyballs, and they resemble the balls used in football (soccer). Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

For purposes of the present invention, the term “fluorescence” refers to the conventional meaning of the term fluorescence, i.e., the emission of light by a substance that has absorbed light or other electromagnetic radiation of a different wavelength.

For purposes of the present invention, the term “fluorescent” refers to any material or mixture of materials that exhibits fluorescence.

For purposes of the present invention, the term “fluorescent dye” refers to any substance or additive that is fluorescent or imparts fluorescence to another material. A fluorescent dye may be organic, inorganic, etc.

For purposes of the present invention, the term “fluorescent microstructure” refers to a microstructure that is fluorescent. A fluorescent microstructure may be made of a naturally fluorescent material or may be made of a nonfluorescent material, such as a phospholipid, doped with a fluorescent dye.

For purposes of the present invention, the term “fluorescent nanostructure” refers to a nanostructure that is fluorescent. A fluorescent nanostructure may be made of a naturally fluorescent material or may be made of a nonfluorescent material, such as a phospholipid, doped with a fluorescent dye.

For purposes of the present invention, the term “fluid” refers to a liquid or a gas.

For purposes of the present invention, the term “freezing by dehydration” refers to removal of residual water content, for instance by incubation in an atmosphere with low water content, for instance a vacuum (<50 mbar) or at relative humidity below 40% (at standard temperature and pressure).

For purpose of the present invention, the term “gel” refers to a solid, jelly-like material that can have properties ranging from soft and weak to hard and tough. On example of a gel is a hydrogel such as: polyethylene glycol, agarose, collagen, pectin, DPPC, etc.

For purposes of the present invention, the term “grating” refers to an array of dots, lines, or a 2D shape that are regularly spaced at a distance that causes coherent scattering of incident light.

For purposes of the present invention, the term “groove” refers to an elongated recess in a stamp. A groove is not limited to a linear groove, unless clearly specified otherwise in the description below. The dimensions of a groove may change depending on the depth of the groove. For example, a groove may be wider at the top of the groove than at the bottom of the groove, such as in a V-shaped groove.

For purposes of the present invention, the term “groove pattern” refers to the pattern made by one or more grooves of a stamp.

For purposes of the present invention, the term “height” refers to the maximum thickness of the microstructure on a substrate, i.e., the maximum distance the microstructure projects above the substrate on which it is located.

For purposes of the present invention, the term “iridescent” refers to any structure that scatters light.

For purposes of the present invention, the term “iridescent microstructure” refers to a microstructure that is iridescent.

For purposes of the present invention, the term “iridescent nanostructure” refers to a nanostructure that is iridescent.

For purposes of the present invention, the term “irregular pattern” refers to a pattern of ridges and recesses that are not organized in a specific geometric pattern. For example, ridges and or recesses printed to resemble a picture of a human face, a picture of a leaf, a picture of an ocean wave, etc. are examples of irregular patterns. Using photolithography, almost any type of pattern for recesses and/or ridges may be formed in a stamp of the present invention.

For purposes of the present invention, the term “light,” unless specified otherwise, refers to any type of electromagnetic radiation. Although, in the embodiments described below, the light that is incident on the gratings or sensors is visible light, the light that is incident on the gratings or sensors of the present invention may be any type of electromagnetic radiation, including infrared light, ultraviolet light, etc., that may be scattered by a grating or sensor. Although, in the embodiments described below, the light that is scattered from the gratings or sensors and detected by a detector is visible light, the light that is scattered by a grating or sensor of the present invention and detected by a detector of the present invention may be any type of electromagnetic radiation, including infrared light, ultraviolet light, etc., that may be scattered by a grating or sensor.

For purposes of the present invention, the term “light source” refers to a source of incident light that is scattered by a grating or sensor of the present invention. In one embodiment of the present invention, a light source may be part of a device of the present invention. In one embodiment a light source may be light present in the environment of a sensor or grating of the present invention. For example, in one embodiment of the present invention a light source may be part of a device that is separate from the device that includes the sensors and detector of the present invention. A light source may even be the ambient light of a room in which a grating or sensor of the present invention is located. Examples of a light source include a laser, a light-emitting diode (LED), an incandescent light bulb, a compact fluorescent light bulb, a fluorescent light bulb, etc.

For purposes of the present invention, the term “line” refers to a “line” as this term is commonly used in the field of nanolithography to refer to a one-dimensional shape.

For purposes of the present invention, the term “lipid” refers to hydrophobic or amphiphilic molecules, including but not limited to biologically derived lipids such as phospholipids, triacylglycerols, fatty acids, cholesterol, or synthetic lipids such as surfactants, organic solvents, oils, etc.

For purposes of the present invention, the term “lipid ink” refers to any material comprising a lipid applied to a stamp.

For purposes of the present invention, the term “lipid multilayer” refers to a lipid coating that is thicker than one molecule.

For purposes of the present invention, the term “lipid multilayer grating” refers to a grating comprising lipid multilayers.

For purposes of the present invention, the term “lipid multilayer structure” refers to a structure comprising one or more lipid multilayers. A lipid multilayer structure may include a dye such as a fluorescent dye.

For purposes of the present invention, the term “liquid continuous medium” refers to a liquid in a system of a dispersion in which dispersed materials are distributed. For example, a solid can be suspended in a liquid continuous medium and droplets of another liquid can also be dispersed in a liquid continuous medium. A “liquid continuous medium” may be a solvent of a solution. For example, a “liquid continuous medium” may be a solvent such as acetone, chloroform, toluene, ethanol, isopropanol, methanol, dichloromethane, acetonitrile, water (anything with a lower boiling point than the solute to be patterned), etc.

For purposes of the present invention, the term “liquid crystal” refers to a distinct phase of matter in a state that has properties between those of conventional liquid states and those of crystalline state (solid). There are many types of liquid crystal states, depending upon the amount of order in the material. For instance, a liquid crystal may flow like a liquid material, but its molecules may be oriented in a crystal-like way. Liquid crystals may be lyotropic liquid crystals such as phospholipids (e.g. 1,2-dioleoyl-sn-glycero-3-phosphocholine [DOPC]), lecithin, sphingomyelin, cardiolipin, Egg PC, thermotropic liquid crystals such as para-azoxyanisole, metallotropic liquid crystals, etc.

For purposes of the present invention, the term “low humidity atmosphere” refers to an atmosphere having a relative humidity of less than 40%.

For purposes of the present invention, the term “lyotropic” refers to the conventional meaning of the term “lyotropic,” i.e., a material that forms liquid crystal phases because of the addition of a solvent.

For purposes of the present invention, the term “microfabrication” refers to the design and/or manufacture of microstructures.

For purposes of the present invention, the term “microparticle” refers to a particle having at least one dimension smaller than 1 mm. A nanoparticle is one type of microparticle.

For purposes of the present invention, the term “microstructure” refers to a structure having at least one dimension smaller than 1 mm. A nanostructure is one type of microstructure.

For purposes of the present invention, the term “multilayer structure” refers to a structure comprising one or more multilayers.

For purposes of the present invention, the term “nanofabrication” refers to the design and/or manufacture of nanostructures.

For purposes of the present invention, the term “nanoparticle” refers to a particle having at least one dimension on the nanoscale, i.e., a dimension between 0.1 and 100 nm.

For purposes of the present invention, the term “nanostructure” refers to a structure having at least one dimension on the nanoscale, i.e., a dimension between 0.1 and 100 nm.

For purposes of the present invention, the term “neat lipid ink” refers to a lipid ink consisting of a single pure lipid ink.

For purposes of the present invention, the term “organic solid” refers to a organic compound whose molecules contain carbon and hydrogen. It may contain any number of other elements such as nitrogen, oxygen, the halogens such as fluorine, chlorine, bromine and iodine, phosphorus, sulfur, etc. An “organic solid” in the present invention may be any of stearic acid, gel-phase phospholipids such as 1,2-dipalmitoyl-sn-glycero-3-phosphocholine (DPPC), biomaterials such as sugars (e.g. cellulose), proteins (e.g. collagen) and nucleic acids (e.g. DNA, RNA), polymers such as PGA (polyglycolide), PLA (polylactic acid), PLGA (poly(lactic-co-glycolide) acid), etc.

For purposes of the present invention, the term “pair of lipid multilayer structures” refers to two nearest neighbor multilayer structures in an array of lipid multilayer structures. In one embodiment of the present invention, a pair of lipid multilayer structures may comprise the same lipid and contain the same drug at the same concentration.

For purposes of the present invention, the term “patterned substrate” refers to a substrate having a patterned array of multilayer structures of one or more dispersed materials on at least one surface of the substrate.

For purposes of the present invention, the term “palette” refers to a substrate having one or more lipid inks that are made available to be picked up or drawn into the recesses or other topographical or chemical features of a stamp. The one or more lipid inks may be located in recesses, inkwells, etc. in the palette, or deposited onto a flat palette.

For purposes of the present invention, the term “palette spot” refers to a single spot of lipid link on a palette. A palette spot may be any shape.

For purposes of the present invention, the term “polymer” refers to a large molecule, or macromolecule, composed of many repeated subunits, known as monomers. Polymers may be synthetic or natural. A “polymer” may be a plastic, a natural or synthetic rubber, etc. A “polymer” may be a hydrogel such as: polyethelene glycol, agarose, collagen, pectinetc.

For purposes of the present invention, the term “plurality” refers to two or more. So an array of microstructures having a “plurality of heights” is an array of microstructures having two or more heights. However, some of the microstructures in an array having a plurality of heights may have the same height.

For purposes of the present invention, the term “recess” refers to a recess of any size or shape in a stamp. A recess may have any cross-sectional shape such as a line, a rectangle, a square, a circle, an oval, etc. The dimensions of a recess may change depending on the depth of the recess. For example, a recess may be wider at the top of the recess than at the bottom of the recess, such as in a V-shaped recess. An example of a recess is a groove.

For purposes of the present invention, the term “recess pattern” refers to the pattern made by one or more recesses of a stamp.

For purposes of the present invention, the term “regular pattern” refers to a pattern of ridges and recesses organized in a specific geometric pattern. For example, a series of parallel recesses and/or lines is one example of a regular pattern. One or more arrays of ridges and recesses arranged in a square, a circle, an oval, a star, etc. is another example of a regular pattern.

For purposes of the present invention, the term “patterned array” refers to an array arranged in a pattern. For example, a patterned array may comprise a single patterned array of one or more dispersed materials. In one embodiment of the present invention, a patterned array may comprise a single patterned array of lipid multilayer structures or two or more patterned arrays of lipid multilayer structures. Examples of patterned arrays of multilayer structures are a patterned array of dots, a patterned array of lines, a patterned array of squares, etc.

For purposes of the present invention, the term “printing” refers to depositing a dispersed material, such as an organic liquid, a lipid ink, on a substrate.

For purposes of the present invention, the term “removing” refers to removing two objects from each other by moving one or both objects away from each other. For example, a stamp may be removed from a palette or substrate by moving the stamp away from the palette or substrate, by moving the palette or substrate away from the stamp or by moving both the stamp and the palette or substrate away from each other.

For purposes of the present invention, the term “ridge” refers to any raised structure. A ridge is not limited to a linear ridge, unless clearly specified otherwise in the description below. A ridge may have any cross-sectional shape such as a line, a rectangle, a square, a circle, an oval, etc. The dimensions of a ridge may change depending on the depth of a neighboring groove. For example, a ridge may be wider at the bottom of the ridge than at the top of the ridge, such as in a V-shaped ridge. A ridge may constitute the entire contacting surface of a stamp after recesses have been formed, etched, etc. into the stamp.

For purposes of the present invention, the term “scattering” and the term “light scattering” refer to the scattering of light by deflection of one or more light rays from a straight path due to the interaction of light with a grating or sensor. One type of interaction of light with a grating or sensor that results in scattering is diffraction.

For purposes of the present invention, the term “sensor” and the term “sensor element” are used interchangeably, unless specified otherwise, and refer to a material that may be used to sense the presence of an analyte.

For purposes of the present invention, the term “square” refers to a microstructure that is square in shape, i.e., has a two-dimensional shape wherein all sides are equal.

For purposes of the present invention, the term “stamped spot” refers to an area of a patterned surface of nanostructures of one or more dispersed materials that originates from a single palette spot on an ink palette used as a source of lipid ink by stamp in depositing the lipid nanostructure. A stamped spot may be any shape.

For purposes of the present invention, the term “stencil” refers one or more structures placed on a substrate to define the shape of an array of multilayer structures of one or more dispersed materials on the substrate using evaporative edge lithography (EEL). A stencil may be one piece or made of several pieces. In one embodiment of the present invention, a stencil may be a single piece with openings in which one or more dipsersions are deposited. In one embodiment of the present invention, a stencil may be several pieces with each piece including openings in which one or more dispersions may be deposited. In one embodiment of the present invention, a stencil may be a set of barriers placed on a substrate in a pattern. In one embodiment, a stencil may be made of an elastomeric material such as polydimethylsiloxane (PDMS).

For purposes of the present invention, the term “strip” refers to a one-dimensional or two-dimensional set of barriers, microstructures, and/or cell cultures. A strip maybe in shape of rectangle. For example, a polydimethylsiloxane (PDMS) barrier in shape of rectangle may also called a PDMS strip.

For purposes of the present invention, the term “surface region” refers to a portion of a surface of a substrate between two multilayer structures or a surface of a substrate surrounded by one or more multilayer structures.

For purposes of the present invention, the term “surround” and the term “surrounded” refer to multilayer structures that surround a region of a substrate or that surround a cell culture in a region of a substrate. For example, lipid multilayer structures that are enclosures may surround a region of a substrate and/or a cell culture in a region of the substrate. The fact that an enclosure may “surround” a cell culture does not mean that the enclosure will prevent the cell culture from migrating across the enclosure. For example, in one embodiment of the present invention, an enclosure comprising a lipid multilayer structure is formed along the edges of an opening in a stencil on a substrate to thereby “surround” a region on the substrate. When a cell culture is deposited in the opening of the substrate, the enclosure will “surround” the cell culture because the walls of the opening of the stencil prevent the cells of the cell culture from being deposited beyond edges of the opening. However, once the stencil is removed, the cells of the cell culture may migrate beyond the enclosure. Also, the when a cell culture is deposited in an opening, some of the cells may be deposited on top of the enclosure.

For purposes of the present invention, the term “topographically structured stamp” refers to a stamp having recesses that form one or more recess patterns. For simplicity, unless specifically indicated otherwise, the term “stamp” refers to a topographically structured stamp.

For purposes of the present invention, the term “toward” refers to decreasing the distance between two aligned objects. For example, a contact controlling positioning device may be used to move: a stamp towards an ink palette, an ink palette towards a stamp, a stamp towards a substrate, a substrate towards a stamp, etc.

### Description

High throughput screening is needed at the early stages of drug discovery. Small molecule microarrays are a promising approach to miniaturizing high throughput screening that could allow tens to hundreds of thousands of compounds to be tested on a single cell culture plate.⁶⁶ With the development of imaging technology, thousands of compounds have been screened for their effect on cellular migration by automated microscopy of scratch assays and by Boyden chamber assays.^{52, 67} Microarrays of different types of lipids have been proposed for molecular screening applications.^{11,12}

Creating biocompatible films with defined features is important to research where these patterned surfaces can give rise to cellular responses such as differentiation, migration, alignment, and other cellular mechanisms. Lipids are used as biocompatible patterning materials to create surface supported monolayers to detect functionality in reconstituted proteins and to measure membrane diffusion. The use of lipids as delivery vectors for delivery of materials to cells is widely studied due to the potential of utilizing them to deliver both lipophilic and hydrophilic drugs and nutrients through liposomes.^{8,9} The hydrophobic nature of some lipids allows them to form structures such as vesicles, liposomes, or membranes in an aqueous environment. Liposomes or vesicles are three-dimensional, self-organized, and nanostructured lipid particles. They are made of the same material as a cell membrane, and therefore, can be filled with drugs and be used as drug- and gene-delivery vehicles.¹⁻⁷

The efficiency of delivery from solution using cationic phospholipids has been extensively studied. Liposomes have been found to enhance the efficacy of anticancer drugs. There is evidence that lipid composition affects cellular uptake and the ability for the drug to kill cancer cells.

Lipid multilayer microstructures and nanostructures are a type of nanomaterial that are effectively multilamellar liposomes confined to a surface. This allows analysis and assays developed for lipid bilayers to be applied to multilayered liposome like structures, which are capable of encapsulating materials. One application of these materials is in the fabrication of small molecule microarrays for drug screening, where drugs encapsulated in the lipid multilayer nanostructures can be delivered to cells cultured on these surfaces for screening of drug efficacy in a microarray format.¹⁰

It has been previously shown that lipid multilayer microarrays with sub-cellular lateral dimensions can be used as a format for delivery of multiple lipophilic anticancer drugs to adherent cells in a microarray format, and measured cytotoxicity as a readout for efficacy.³⁴ These arrays are capable of encapsulating drugs or drug candidates in an organic phase and preventing them from leaking into solution, yet allowing uptake by cells. Importantly, lipid multilayer microarrays are compatible with lipophilic compounds, while other drug screening microarrays are either limited to water soluble compounds that diffuse out of a gel into water,⁶⁸ or must be covalently linked to the surface and cannot be taken up by the cells.⁶⁹ Most drug candidates at the early stages of drug discovery have low water solubility, which is quantified by a high octanol to water partition coefficient (Log P). In standard high throughput screening, dimethyl sulfoxide (DMSO) is used as solvent to deliver compounds with high Log P values in water,⁷⁰ yet DMSO can have undesirable effects on certain cell-based assays.^{71, 72} More importantly, DMSO cannot be used to deliver lipophilic drugs to cells from microarrays.

Methods previously developed to fabricate lipid multilayer patterns include dip-pen nanolithography (DPN),¹⁵⁻¹⁷ soft lithography (e.g. micro-contact printing),^{73,22} photothermal patterning,²⁰ and capillary assembly.⁷⁴

Dip-pen nanolithography (DPN) is a method that uses an atomic force microscopy (AFM) tip to deliver materials directly to a specific region of a target substrate. DPN can be carried out with parallel tip arrays for large area fabrication.^{30,31} Multiple materials can be simultaneously delivered to a surface from different tips in parallel arrays, for instance, using microfluidic channels to ink the tips, or microarray technology to deliver the different lipid inks to the AFM tips. It can fabricate arbitrary structures from a variety of molecular inks.²⁶⁻²⁹ The use of masks is not required, and sub-100-nm resolution can be achieved.²⁹ Similar approaches to nanosurface and microsurface patterning include soft lithography³² and polymer pen lithography.³³

The present inventors have established the concept of using surface supported multilayers as carriers for lipophilic cancer drugs to cells.³⁴ The feasibility of delivery of materials to cells from lipid multilayer patterns created with dip-pen nanolithography (DPN) has been established. When carried out with lipid-based inks, DPN is capable of forming lipid multilayer nanostructures, where the multilayer thickness can be controlled. Multilayer thickness is particularly important for delivery applications because it allows encapsulation of materials such as drug candidates within the multilayers.

Although DPN is well suited for prototype fabrication due to its ability to directly write arbitrary patterns, there are currently practical limits to its scalability for multi-material patterning. For instance, for small molecule microarray applications in drug screening, it would be desirable to have hundreds of thousands of different small molecules integrated onto a single surface. The ability for DPN to multiplex has been demonstrated for 24 different lipid inks,³⁵ but the scalability of that process has yet to be shown.

Lipid multilayer stamping uses a structured polymeric stamp to print lipid multilayer structures onto a surface.²² It combines several aspects of well-established microfabrication methods in a new approach that is uniquely suited for lipid multilayer nanofabrication. In particular, lipid multilayer stamping combines the lateral patterning capabilities and scalability of microcontact printing,³² with the topographical control of nanoimprint lithography³⁶ to create nanostructured lipid multilayer arrays. A disadvantage of lipid multilayer stamping is that it requires pre-fabrication of a master, necessitating DPN to identify the optimal stamp geometry. Once that is determined, lipid multilayer stamping is a scalable method capable of mass production of lipid multilayer microarrays.

The migration of cells collectively is an important aspect of cancer metastasis,^{41,42} angiogenesis,^{43,44} wound healing,⁴⁵ and organismal development.^{46, 47} Cell migration can be observed in vitro using tissue culture. A variety of in vitro systems have been developed to assess the effects of compounds and microenvironmental conditions on the migration of cells in culture.^{84, 49} Commonly used methods include “scratch-wound” assay,^{48, 50} removable fencing assays,⁵¹ Boyden chamber assays,⁵² biodegradable barriers,^{53, 54} and microfluidic techniques.⁵⁵⁻⁵⁷ These methods have the common feature that cells are cultured on a certain part of a two-dimensional substrate or three-dimensional volume and allowed to migrate into a region without cells. The number or speed of individual or collectively migrating cells into the unpopulated regions is then measured.

Because it is simple and cheap, the “scratch-wound” assay is most commonly used to measure basic cell migration parameters such as speed, persistence, and polarity.⁴⁸ In this assay, a cell monolayer is grown to confluence and a cell-free area is then created often by scraping away the cells with a pipette tip or other object. Cells at the wound edge migrate into the wound space. The cells are then observed with microscopy to measure the number and rate at which the scraped area is repopulated. This assay generates a strong directional migratory response. Drawbacks of this assay, however, are the inconsistent size and placement of the wound area within the cell culture monolayer and the mechanical damage to the edge layer of cells and the substrate surface.⁵⁸ As a result, although this method has been adequate for qualitative characterization of migration inhibitors, it does not provide the highly reproducible results required for quantitative compound structure-activity relationship evaluation.

The removable fencing assay reported by Lenhert et al. provides an alternative to the scratch assay.¹¹ In this assay, cell growth was initially confined by a barrier to a 5 mm diameter area on a substrate for 2 days; the barrier was then removed; images of cell cultures were taken periodically after the cells were allowed to move freely onto areas previously uncovered by cell cultures on the substrate.¹¹ In similar assays using the commercially available Oris™ kit (Platypus Technologies), silicon cell seeding stoppers are used as barriers and are placed at the center of each well in a 96-well plates to thereby form a detection zone.⁵⁴ This assay allows the formation of precisely placed and homogeneously sized cell-free areas within the monolayer into which migration can occur without releasing factors from damaged or dead cells.⁵⁴

In Boyden chamber assays,⁵² a cylindrical cell culture insert is nested inside the well of a cell culture plate. The insert contains a polycarbonate membrane at the bottom with pores in defined sizes. Cells are seeded in the top of the insert in serum-free media, while serum or similarchemoattractants are placed in the well below. Migratory cells move through the pores toward the chemoattractant below and can be stained or quantified in a plate reader.

Microfluidic fabrication technologies have been used to study cellular migration within the past decade. These technologies were developed to precisely and simultaneously control multiple environmental factors such as biochemical and biomechanical forces.^{55, 59-62} For examples, Huang et al. have used a microfluidic-based migration assay to induce wounds by partially detaching a confluent monolayer using laminar flows in the presence of trypsin;⁵⁷ Kwak et al. created a device using optical microscopy assisted with computer software to collect data on cell size, migration path, distance and speed to thereby record individual Human Umbilical Vein Endothelial Cell (HUVEC) and NCI-H23 cell migration in real-time.⁶³ Other methods have also been described to track individual cells within a confluent population to demonstrate contact inhibition in Madin-Darby canine kidney (MDCK) epithelial cells or to gather information on individual cell migration in 3D models.^{64, 65}

Current methods to analyze cellular migration are limited by the number and type of different compounds and dosages that can be tested in parallel.

This present invention proposes a technology called evaporative edge lithography (EEL). Through the EEL technology, one or more dispersed materials can be deposited on a surface of a substrate and form one or more patterned material depositions on the surface of the substrate along one or more respective edges of one or more pairs of barriers, or along one or more respective peripheral edges of one or more respective openings in a stencil. As shown in some examples in the present application, a patterned material deposition may encompass one or more multilayer structures, for example, multilayer nanostructures. In some embodiments of the present invention, the one or more patterned material depositions are along one or more respective edges inside one or more respective spaces between one or more pairs of barriers. In another embodiment, the one or more patterned material depositions may be formed along one or more respective edges outside the one or more spaces between one or more pairs of barriers.

One of the advantages of EEL over other technologies such as DPN is its compatibility with a variety of materials. One or more dispersions that encompass one or more types of liquid continuous media and one or more types of dispersed materials can be used to form the patterned material depositions on a surface of a substrate through the technology of EEL. Preferably, the liquid continuous medium is volatile. In some embodiments of the present invention, the patterned material depositions may encompass dispersed materials such as: organic liquids, organic solids, gels, liquid crystals, metallic nanoparticles, semiconductor nanoparticles such quantum dots, microscopic beads, etc.

Disclosed embodiments of the present invention also provide an edge evaporation lithography (EEL) method to fabricate lipid-based drug delivery microarrays. Evaporation induced self-assembly (EISA) is a technique based on the evaporation of a solution containing precursors to be assembled. EISA has been used previously with lipid membranes.^{75,76} In EEL, a set of elastomeric barriers or stencils directs the precipitation of lipid and drug solutes along an edge to therefore form a drug-encapsulated lipid multilayer line. Drugs, including lipophilic drugs, can be encapsulated in the lipid multilayer line and be delivered to adherent cells for various assays including migration assays. Unlike other migration assays, this approach makes it possible to screen different compounds and dosages on the same surface, with scalability for high throughput screening microarrays.

In one embodiment of the present invention, a barrier may comprise a strip. In one embodiment of the present invention, a strip as a barrier may comprise a polydimethylsiloxane (PDMS) strip. In one embodiment of the present invention, a PDMS strip as a barrier may be approximately 15 micrometers in length and approximately 1 micrometer in width.

In one embodiment, an elastomeric stencil directs the precipitation of lipid and drug solutes (e.g. docetaxel) along an edge resulting in a drug-encapsulated lipid multilayer line that can deliver lipophilic drugs to adherent cells for migration assays. The thickness of these lipid films is controlled, which results in controlling the dosage of material that is taken up by cells cultured over these areas.

In one embodiment, the present invention provides a migration assay that allows for local delivery of multiple different dosages of the lipophilic drug docetaxel to cells in a microarray format. For this purpose, lipid multilayers are fabricated with solutions of lipid and drugs that can be taken up by adherent HeLa cells cultured on the lipid multilayer arrays. Upon removal of the stencil, migration can be assayed much like a fencing assay, yet in a microarray format. Lipid patterning along the edges is found to be crucial for this assay because cells do not adhere to the lipid multilayer coated surfaces when the entire spaces bounded by barriers are filled with lipid multilayer. Our results demonstrate the assays compatibility with the lipophilic drug docetaxel, which was locally delivered to adherent HeLa cells from microarrays at various dosages in a way that allows the measurement of cell migration inhibition. Unlike other migration assays, this approach makes it possible to screen different compounds and dosages on the same surface and is suitable for high throughput screening microarrays. Additionally, because of the precipitation properties, the drug or small molecules encapsulated will only be delivered to cells at the edge of the stencil. As a result, the drug will selectively affect the migrating cells but not non-migratory cells only at the edge of the barriers.

In one embodiment, the present invention provides a method and apparatus for preventing cross-contamination of lipid-encapsulated materials in arrays.

In one embodiment, the present invention provides a method and apparatus for assaying for cell response to materials delivered from the microarray.

In one embodiment, the present invention provides a method for assaying the efficacy of lipid multilayer delivered drugs to leukemia cells.

In one embodiment, the present invention provides a method and apparatus for monitoring and controlling cell migration using fluorescently labeled lipid multilayers.

The patterned substrates of the present invention may be used in a variety of cellular assay methods. In one embodiment, an assay method of the present invention comprises the following steps: (1) Cells are seeded on the array; (2) Cells are allowed to grow; (3) The cells are stained (optional); and (4) Cells are counted and the number of cells on each spot is used as a measure of viability. In other embodiments of the present invention, steps 3 and 4 are replaced by second messenger assays, reporter gene assays, or high content screening methods.

In one embodiment, the present invention provides a microarray-based migration assay that combines fencing with lipid multilayer drug delivery in vitro. For this purpose, the present invention provides a lipid multilayer fabrication method, i.e., edge evaporation lithography (EEL), that is capable of producing linear lipid multilayer nanostructures along the edge of a stencil. This method makes use of capillary assembly onto a pre-patterned surface in a way similar to that carried out by Diguet et al.,⁷⁴ with a difference being that EEL uses an edge between a stencil and a surface as a one-dimensional template rather than controlled evaporation on a chemically patterned surface.

In one embodiment of the present invention, such microarrays may be suitable for the investigation of the effect of the antimicrotubule agent docetaxel on HeLa cell migration. Results demonstrate in vitro that docetaxel delivered into the cells locally from surface supported lipid films significantly inhibit cellular migration.

In one embodiment of the present invention, microarrays produced by EEL techniques may be used to study of the effects of poorly water soluble drugs, i.e., hydrophobic drugs, on cell migration, structures and function.

In one embodiment of the present invention, microarrays produced by EEL techniques may be used to study of the effects of poorly water soluble drugs, i.e., hydrophobic drugs, on cell migration, structures and function.

In one embodiment of the present invention, microarrays produced by EEL techniques may be used to study of the effects of water soluble drugs, i.e., hydrophilic drugs, on cell migration, structures and function.

In one embodiment of the present invention, microarrays produced by EEL techniques may be used for in vitro screening of a variety of different drugs for their effects on cells. This migration assay is unique in that multiple different compounds and dosages can be screened on the same surface, suitable for high throughput screening microarrays.

In one embodiment of the present invention, evaporative edge lithography may be used to produce linear lipid multilayer nanostructures along the edge of a stencil. The elastomeric stencil directs the precipitation of lipid and drug solutes (e.g. docetaxel) along an edge resulting in a drug-encapsulated lipid multilayer line that can deliver lipophilic drugs to adherent cells for migration assays. The thickness of these lipid films may be controlled to thereby control the dosage of material that is taken up by cells cultured over these areas. This is advantageous because unlike other migration assays, this approach makes it possible to screen different compounds and dosages on the same surface, with scalability for high throughput screening microarrays to assay for cell migration.

In one embodiment of the present invention, a drug or small molecules encapsulated within the lipid multilayer nanostructures may be delivered to cells only at the edge of the stencil because of the precipitation properties which can be important to selectively affect the migrating cells at the edge from non-migratory cells.

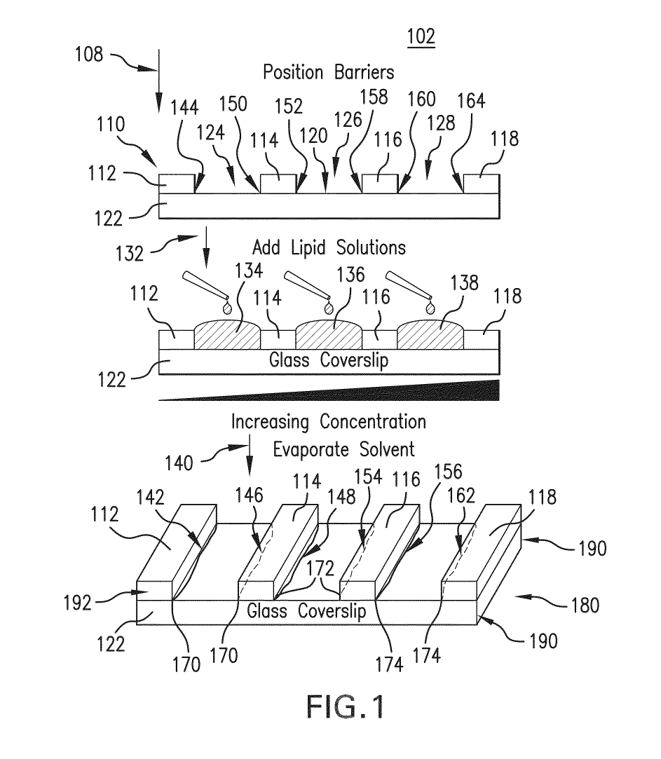

FIG. 1 shows an edge evaporation lithography (EEL) method 102 to fabricate a lipid-based drug delivery microarrays 180 according to one embodiment of the present invention. At step 108 a set of polydimethylsiloxane (PMDS) barriers 112, 114, 116 and 118 is placed on a surface 120 of a substrate 122 to thereby form a space 124 between barriers 112 and 114, a space 126 between barriers 114 and 116, and a space 128 between barriers 116 and 118. At step 132 spaces 124, 126 and 128 are filled with lipid solutions 134, 136 and 138, respectively, comprising a solvent containing one or more lipids mixed with a drug. The drug is present in solutions 134, 136 and 138 at three different concentrations. The concentration of the drug is higher in solution 136 than in solution 134. The concentration of the drug is higher in solution 138 than in solution 136. At step 140, the solvent is evaporated from solutions 134, 136 and 138 to thereby form lipid multilayer structure 142 along edge 144 of barrier 112, lipid multilayer structures 146 and 148 along edges 150 and 152 of barrier 114, lipid multilayer structures 154 and 156 along edges 158 and 160 of barrier 116, and lipid multilayer structure 162 along edge 164 of barrier 118. Lipid multilayer structures 142 and 146 are a pair of lipid multilayer structures having the same drug concentration. Lipid multilayer structures 148 and 154 are a pair of lipid multilayer structures having the same drug concentration. Lipid multilayer structures 156 and 162 form a pair of lipid multilayer structures having the same drug concentration. Cells can be seeded on surface regions 170, 172, and 174 of respective spaces 124, 126 and 128. Fabricated lipid-based drug delivery microarrays 180 are suitable for various assays, including high throughput screening of drugs for variety of effects on cells.

In the embodiment shown in FIG. 1, sufficient surface adhesion exists between substrate 122 and lipid solutions 134, 136 and 138, and between barriers 112, 114, 116 and 118 and lipid solutions 134, 136 and 138. Therefore, the lipid solution does not run off edges of 190 of substrate 122 between barriers 112, 114, 116 and 118. Ends 192 of barriers 112, 114, 116 and 118 are approximately flush with edges of substrate 122 in the embodiment shown in FIG. 1. But in some embodiments of the present invention, the ends of the barriers may not extend to the edges of the substrate.

In one embodiment of the present invention, the substrate of FIG. 1 may be a glass coverslip.

In another embodiment of the present invention, the substrate of FIG. 1 may be a glass slide.

For simplicity of illustration, in FIG. 1, the microarray has three pairs of lipid multilayer structures having the same drug concentration. However, there may be any number of pairs of lipid multilayer structures having the same drug concentration in a microarray.

In one embodiment of the present invention, the plurality of lipid solutions comprises one or more drugs, and wherein each lipid multilayer structure of the lipid multilayer structures is a microstructure comprising the one or more lipids and the one or more drugs of one lipid solution of the plurality of lipid solutions.

In one embodiment, the present invention a first array of one or more arrays of lipid multilayer structures comprise lipid multilayer structures containing a first drug and wherein a second array of the one or more arrays comprise lipid microstructures comprising a second drug different from the first drug.

In one embodiment of the present invention, at least one array of lipid multilayer structures of the one or more arrays of lipid multilayer structures comprises a first pair of nearest neighbor lipid multilayer structures comprising a first drug at a first concentration and second pair of nearest neighbor lipid multilayer structures comprising the first drug at a second concentration that is different from the first concentration.

Although FIG. 1 shows an edge evaporation lithography (EEL) method for fabricating a lipid microarray using a dispersed material encompassing one or more lipids, the techniques shown in FIG. 1 may be used with other types of dispersed materials to form microarrays of these other types of dispersed materials. For example, barriers may be used to form microarrays of dispersed materials such as: organic liquids, organic solids, gels, liquid crystals, metallic nanoparticles, semiconductor nanoparticles such quantum dots, microscopic beads, etc.

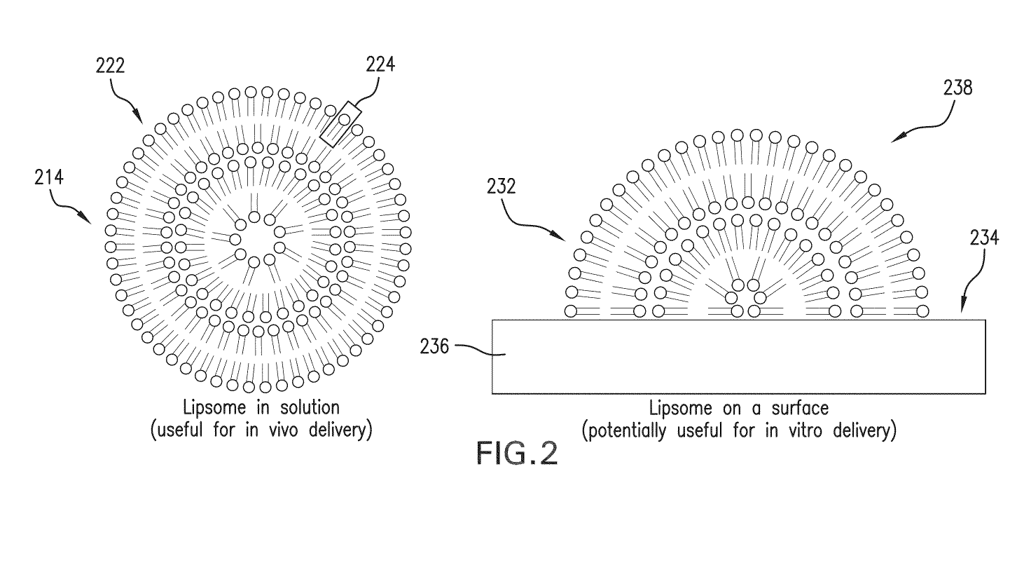

FIG. 2 shows one possible supramolecular structure of a liposome and a surface-supported lipid nanostructure. A multilamellar liposome 222 self-assembles in solution 214. Multilamellar liposome 222 is comprised of a typical phospholipid 224. A surface-supported lipid nanostructure 238 comprises a loaded a lipid multilayer liposome 232 on a surface 234 of a substrate 236.

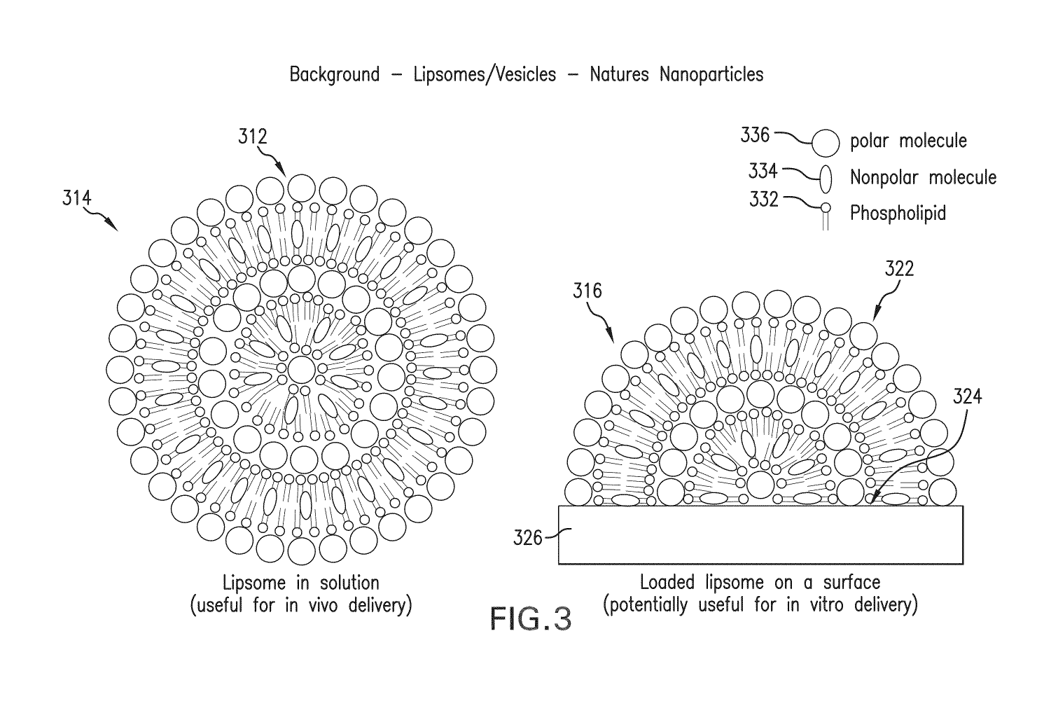

FIG. 3 shows one possible supramolecular structure of a loaded liposome 312 in a solution 314 and a loaded surface-supported lipid nanostructure 316. Loaded surface-supported lipid nanostructure 316 comprises a loaded liposome 322 on a surface 324 of a substrate 326. Loaded liposome 312 and loaded surface-supported lipid nanostructure 316 are comprised of phospholipids 332, nonpolar molecules 334, and polar molecules 336. FIG. 3 shows possible locations of encapsulated materials within both solution-based liposomes and surface-supported liposomes or lipid multilayer nanostructures.

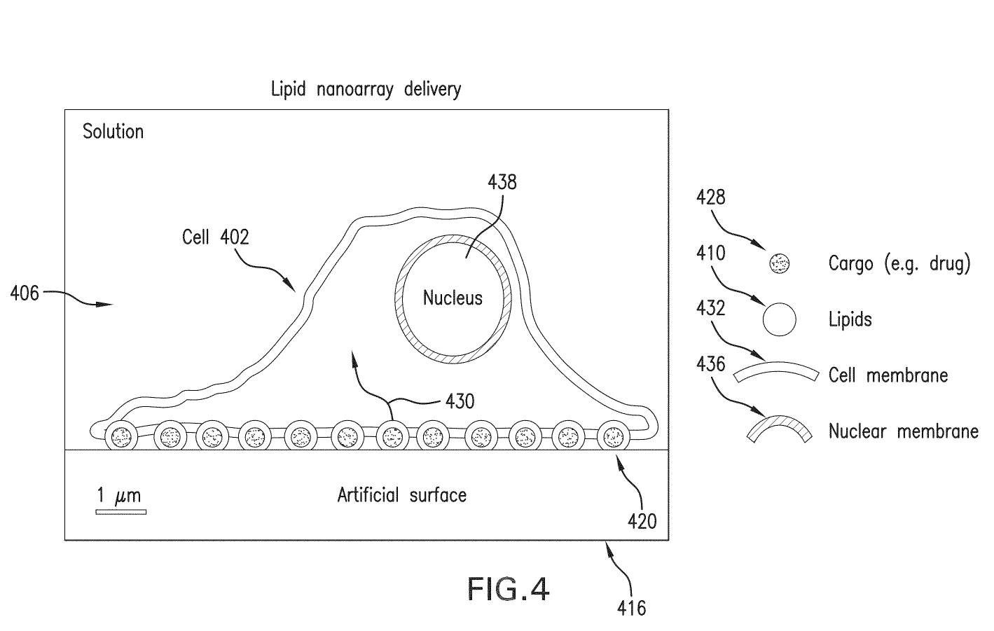

FIG. 4 shows a lipid nanoarray delivery involving a cell 402, a solution (or in some embodiments a gas) 406, an artificial surface 416, a drug 428, lipids 410, and a cell membrane 432. Cell 402 includes a nucleus 438 having a nuclear membrane 436. Lipids 410 are present as lipid multilayer nanostructures 420 on an artificial surface 416. Lipid multilayer nanostructures 420 are in contact with cell 402 thereby allowing drug 428 encapsulated in each lipid multilayer nanostructures 420 to be taken into cell 402 by endocytosis of each lipid multilayer nanostructure 420 as shown by arrow 430, allowing control of both dosage and the possibility to deliver different materials as to different cells in the same solution or environment.

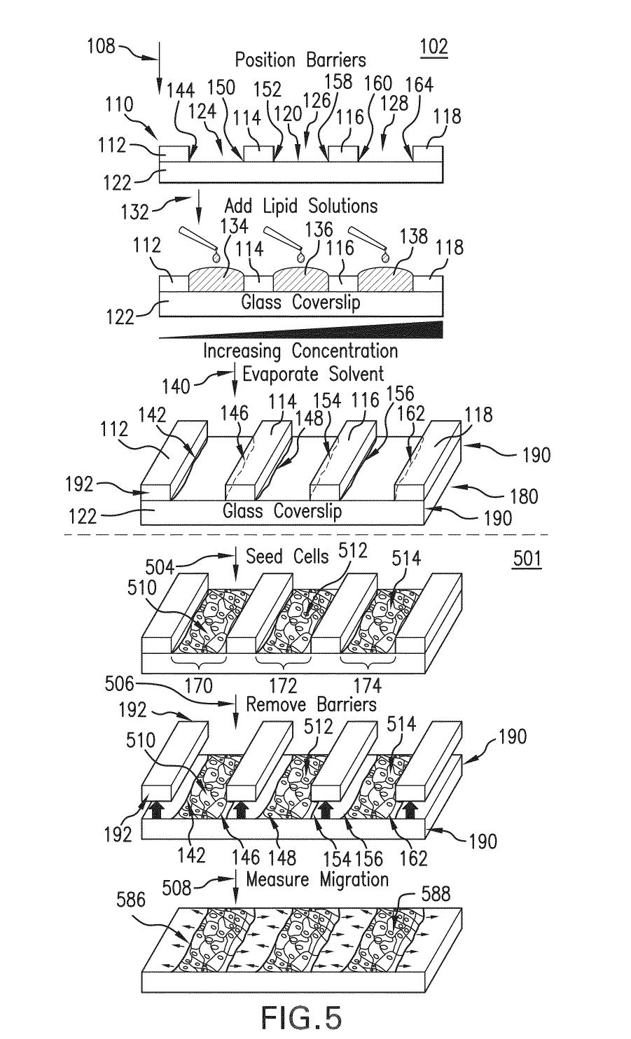

FIG. 5 shows an edge evaporation lithography (EEL) method of a liposomal drug microarray 501 for cell migration assays according to one embodiment of the present invention.

At step 504, cell cultures 510, 512 and 514 respectively are seeded on surface regions 170, 172, and 174 of respective spaces 124, 126 and 128 of lipid-based drug delivery microarrays 180 fabricated according to one embodiment of the present invention shown in FIG. 1. Surface regions 170, 172, and 174 are three portions of surface 120 of substrate 122. Cell cultures 510, 512, and 514 are then cultured to confluence, i.e., to fill respective surface regions 170, 172, and 174.

At step 506, barriers 112, 114, 116 and 118 are removed. Cell cultures 510 forms an array of cell cultures which are bounded on two sides by a pair of nearest neighbor lipid multilayer structures, i.e., lipid multilayer structures 142 and 146. Cell cultures 512 forms an array of cell cultures which are bounded on two sides by a pair of nearest neighbor lipid multilayer structures, i.e., lipid multilayer structures 148 and 154. Cell cultures 514 forms an array of cell cultures which are bounded on two sides by a pair of nearest neighbor lipid multilayer structures, i.e., lipid multilayer structures 156 and 162.

At step 508, the migrations of the cells of cell cultures 510, 512, and 514 are measured. Each of lipid multilayer structures 142, 146, 148, 154, 156 and 162 is microstructure and they together form a lipid multilayer microarray 586. Cell cultures 510, 512 and 514 together form cell culture arrays 588. Prior to cell migration, each array of cell culture arrays 588 is bounded on two sides by two lipid multilayer structures of lipid multilayer microarray 586. Once barriers 112, 114, 116 and 118 are removed, the cells of cell culture arrays 510, 512 and 514 are free to migrate across lipid multilayer structures 142, 146, 148, 154, 156 and 162.

FIG. 5 shows only one embodiment of the edge evaporation lithography (EEL) method for fabricating a lipid microarray using a dispersed material encompassing one or more lipids. The EEL techniques illustrated in FIG. 5 may be used with other types of dispersed materials to form microarrays of these other types of dispersed materials. For example, barriers may be used to form microarrays of dispersed materials such as: organic liquids, organic solids, gels, liquid crystals, metallic nanoparticles, semiconductor nanoparticles such quantum dots, microscopic beads, etc. In other embodiments, microarray of lipid multilayer structures may be formed for other purposes.

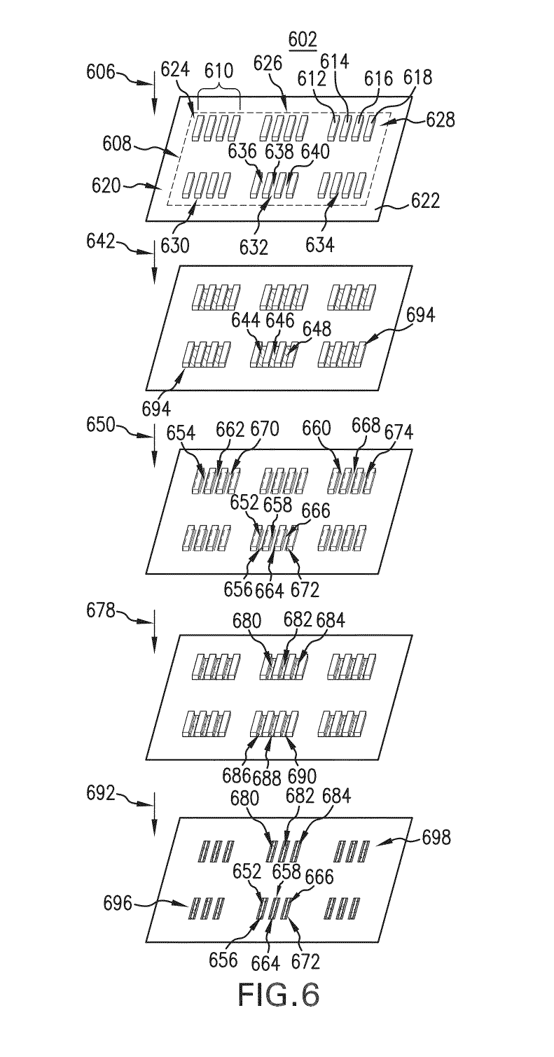

FIG. 6 shows an edge evaporation lithography (EEL) method 602 according to one embodiment of the present invention. At step 606 a stencil 608 comprising six sets 610 of polydimethylsiloxane (PDMS) strips, 612, 614, 616 and 618 is formed on a surface 620 of a substrate 622 at six positions, i.e., position 624, position 626, position 628, position 630, position 632 and position 634. Between barriers 612 and 614 is a space 636, between barriers 614 and 616 is a space 638, between barriers 616 and 618 is a space 640. At step 642, spaces 636, 638 and 640 at each of the six positions 624, 626, 628, 630, 632, and 634 on substrate 622 are filled with lipid solutions 644, 646 and 648, respectively. Lipid solutions 644, 646 and 648 each comprises a solvent containing one or more lipids mixed with a drug. The concentration of the drug is higher in lipid solution 646 than in lipid solution 644 and is higher in 648 than in 646. A different drug is used in lipid solutions 644, 646 and 648, for example, at each of the six positions 624, 626, 628, 630, 632 and 634 on substrate 622.

At step 650, the solvent is evaporated from lipid solutions 644, 646 and 648 to thereby form a lipid multilayer structure 652 along edge 654 of barrier 612, lipid multilayer structures 656 and 658 along edges 660 and 662 of barrier 614, lipid multilayer structures 664 and 666 along edges 668 and 670 of barrier 616, and a lipid multilayer structure 672 along edge 674 of barrier 618. Lipid multilayer structures 652 and 656 are a pair of lipid multilayer structures having the same drug concentration. Lipid multilayer structures 658 and 664 are a pair of lipid multilayer structures having the same drug concentration. Lipid multilayer structures 666 and 672 form a pair of lipid multilayer structures having the same drug concentration.

Lipid multilayer structures 652, 656, 658, 664, 666 and 672 are each microstructures. Each set of lipid multilayer structures 652, 656, 658, 664, 666 and 672 at each of the six positions 624, 626, 628, 630, 632 and 634 on substrate 622 together form a microarray 696. The microarrays 696 at the six positions 624, 626, 628, 630, 632 and 634 on substrate 622 together form a microarray of lipid multilayer structures.

At step 678, cell cultures 680, 682 and 684, respectively are seeded on surface regions 686, 688 and 690 of respective spaces 636, 638 and 640 at each of the six positions 624, 626, 628, 630, 632 and 634 on substrate 622. Cell cultures 680, 682 and 684 at each of the six positions 624, 626, 628, 630, 632 and 634 on substrate 622 are then cultured to confluence, i.e. to fill respective spaces 636, 638 and 640 at each of the six positions 624, 626, 628, 630, 632 and 634 on substrate 622.

At step 692, barriers 612, 614, 616 and 618 are removed from the six positions 624, 626, 628, 630, 632 and 634 on surface 620 of substrate 622. Each set of cell cultures 680, 682 and 684 at each of the six positions 624, 626, 628, 630, 632 and 634 on substrate 622 together form arrays 698 of cell cultures. Prior to cell migration, each cell culture of the arrays 698 of cell cultures is bounded on two sides by two lipid multilayer structures. At each of the six positions 624, 626, 628, 630, 632 and 634 on substrate 622, cell culture 680 is bounded on two sides by a pair of nearest neighbor lipid multilayer structures, i.e., lipid multilayer structures 652 and 656. At each of the six positions 624, 626, 628, 630, 632 and 634 on substrate 622, cell culture 682 is bounded on two sides by a pair of nearest neighbor lipid multilayer structures, i.e., lipid multilayer structures 658 and 664. At each of the six positions 624, 626, 628, 630, 632 and 634 on substrate 622, cell culture 684 is bounded on two sides by a pair of nearest neighbor lipid multilayer structures, i.e., lipid multilayer structures 666 and 672.

Once barriers 612, 614, 616 and 618 are removed, the cells of cell cultures 680, 682 and 684 are free to migrate across lipid multilayer structures 652, 656, 658, 664, 666 and 672. The migration rates of the cells at each of the six positions 624, 626, 628, 630, 632 and 634 on substrate 622 may be measured, thereby allowing six different drugs to be assayed simultaneously.

In FIG. 6, there is sufficient surface adhesion between substrate 622 and lipid solutions 644, 646 and 648 at each of the six positions 624, 626, 628, 630, 632 and 634 on substrate 622 and between barriers 612, 614, 616 and 618 and lipid solutions lipid solutions 644, 646 and 648 at each of the six positions 624, 626, 628, 630, 632 and 634 on substrate 622 so that lipid solutions lipid solutions 644, 646 and 648 does not substantially disperse beyond ends 694 of barriers 612, 614, 616 and 618.

For simplicity of illustration, in FIG. 6, each microarray at each of the six positions 624, 626, 628, 630, 632 and 634 on substrate 622 has three pairs of lipid multilayer structures. However, there may be any number of pairs of lipid multilayer structures at each of the six positions 624, 626, 628, 630, 632 and 634 on substrate 622. There may be any number of positions on a surface of a substrate.

FIG. 6 shows only one embodiment of the edge evaporation lithography (EEL) method for fabricating a lipid microarray using a dispersed material encompassing one or more lipids. The techniques shown in FIG. 6 may be used with other types of dispersed materials to form microarrays of these other types of dispersed materials. For example, barriers may be used to form microarrays of dispersed materials such as: organic liquids, organic solids, gels, liquid crystals, metallic nanoparticles, semiconductor nanoparticles such quantum dots, microscopic beads, etc. In other embodiments, microarray of lipid multilayer structures may be formed for other purposes.

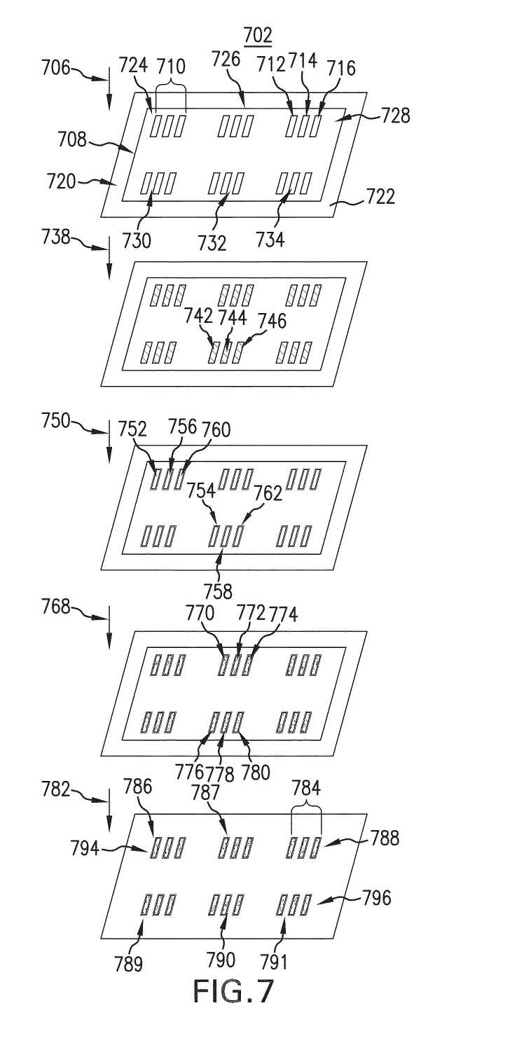

FIG. 7 shows an edge evaporation lithography (EEL) method 702 according to one embodiment of the present invention. At step 706 a stencil 708 comprising six sets 710 of openings 712, 714 and 716 are formed on a surface 720 of a substrate 722. Although for clarity of illustration, stencil 708 is shown as being substantially two-dimensional in FIG. 7, stencil 708 is actually three-dimensional and has a thickness similar to the thickness of the barriers shown in FIGS. 1 and 6. Sets 710 of openings 712, 714 and 716 are at six positions on stencil 708, i.e., position 724, position 726, position 728, position 730, position 732 and position 734. At step 738, openings 712, 714 and 716 at each of the six positions 724, 726, 728, 730, 732, and 734 on substrate 722 are filled with lipid solutions 742, 744 and 746, respectively. Lipid solutions 742, 744 and 746 each comprises a solvent containing one or more lipids mixed with a drug. The concentration of the drug is higher in lipid solution 744 than in lipid solution 742 and is higher in 746 than in lipid solution 744. A different drug is used, for example, in lipid solutions 744, 746 and 748 at each of the six positions 724, 726, 728, 730, 732, and 734 on substrate 722.

At step 750, the solvent is evaporated from lipid solutions 742, 744 and 746 to thereby form lipid multilayer structure 752 along peripheral edge 754 of opening 712, lipid multilayer structure 756 along peripheral edge 758 of opening 714, and lipid multilayer structure 760 along peripheral edge 762 of opening 716. Each of lipid multilayer structures 752, 756 and 760 is an enclosure.

At step 768, cell cultures 770, 772 and 774, respectively are seeded on surface regions 776, 778 and 780 surrounded by lipid multilayer structures, 752, 756 and 760, respectively. Surface regions 776, 778 and 780 are three portions of surface 720 of substrate 722. Cell cultures 770, 772 and 774 are then cultured to confluence, i.e. to fill surface regions 776, 778 and 780.

At step 782, stencil 708 is removed from substrate 722 to thereby form six arrays 784 of lipid multilayer structures 752, 756 and 760. Each lipid multilayer structure 752 surrounds a cell culture 770, each lipid multilayer structure 756 surrounds a cell culture 772, and each lipid multilayer structure 760 surrounds a cell culture 774. Arrays 784 are at six positions on substrate 722, i.e., positions 786, 787, 788, 789, 790 and 791.

Once stencil 708 is removed from substrate 722, cells of respective cell cultures 770, 772 and 774 are free to migrate across lipid multilayer structures 752, 756 and 760. The migration rates of the cells at each of the six different positions may be measured, thereby allowing six different drugs to be assayed simultaneously.

Lipid multilayer structures 752, 756 and 760 are each microstructures and therefore, arrays 784 are microarrays. Together, the six arrays 784 together form an array 794 of microstructures.

Each set of cell cultures 770, 772 and 774 at each of the six positions 724, 726, 728, 730, 732, and 734 on substrate 722 together forms arrays 796 of cell cultures. The arrays of cell cultures at the six positions 724, 726, 728, 730, 732, and 734 on substrate 722 together form arrays of cell cultures over substrate 722.

For simplicity of illustration, in FIG. 7, each microarray at each of the six positions 724, 726, 728, 730, 732, and 734 on substrate 722 has three lipid multilayer structures. However, there may be any number of lipid multilayer structures at each of the six positions 724, 726, 728, 730, 732, and 734 on substrate 722. There may be any number of positions on a surface of a substrate.

The substrate used in the present invention may be any conventional substrate material used in cellular assays such as glass, functionalized glass, polystyrene, polymethylmethacralate, etc. In one embodiment the substrate may be tissue culture plastic, i.e. a cell culture microplate.

Although peripheral edges of openings 712, 714, and 716 on stencil 708 shown in FIG. 7 are linear, a barrier may be linear or non-linear. A barrier may comprise a stencil having openings in various shapes, including having an edge of an opening in non-linear.

FIG. 7 shows only one embodiment of the edge evaporation lithography (EEL) method for fabricating a lipid microarray using a dispersed material encompassing one or more lipids. The techniques shown in FIG. 7 may be used with other types of dispersed materials to form microarrays of these other types of dispersed materials. For example, barriers may be used to form microarrays of dispersed materials such as: organic liquids, organic solids, gels, liquid crystals, metallic nanoparticles, semiconductor nanoparticles such quantum dots, microscopic beads, etc. In other embodiments, microarray of lipid multilayer structures may be formed for other purposes.

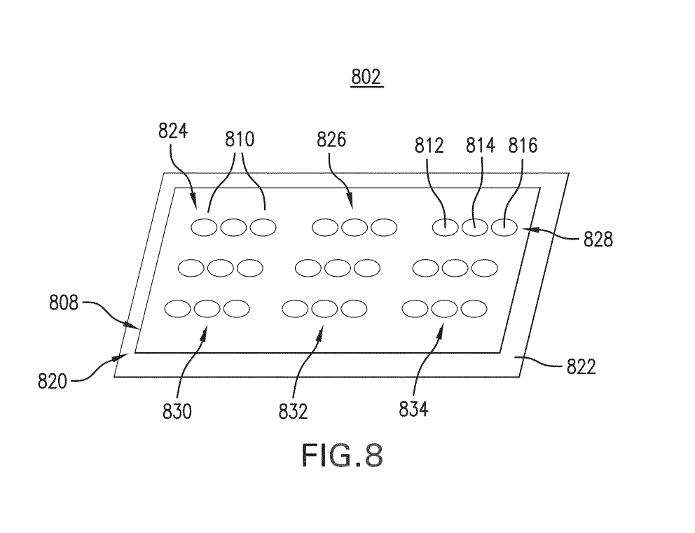

FIG. 8 shows a stencil 802 having openings with non-linear edges, for example, the circular edges, according to one embodiment of the present invention. Stencil 802 comprising six sets 810 of openings 812, 814 and 816 are formed on a surface 820 of a substrate 822. Each of the six sets 810 of openings has a circular peripheral edge. Although for clarity of illustration, stencil 802 is shown as being substantially two-dimensional in FIG. 8, stencil 802 is actually three-dimensional and has a thickness similar to the thickness of barriers shown in FIGS. 1 and 6. Sets 810 of openings 812, 814 and 816 are at six positions on stencil 802, i.e., position 824, position 826, position 828, position 830, position 832 and position 834. Lipid multilayer microarrays at the six positions 824, 826, 828, 830, 832, and 834 on substrate 822 can be fabricated by following the steps illustrated in FIG. 7. Each lipid multilayer microarray, for example, at each of the six positions 824, 826, 828, 830, 832, and 834 on substrate 822 would be three lipid multilayer structures in circle.

For simplicity of illustration, in FIG. 8, each set of the six sets of openings has three openings at each of the six positions 824, 826, 828, 830, 832, and 834 on substrate 822. However, there may be any number of openings at each of the six positions 824, 826, 828, 830, 832, and 834 on substrate 822. There may be any number of positions on a surface of a substrate.

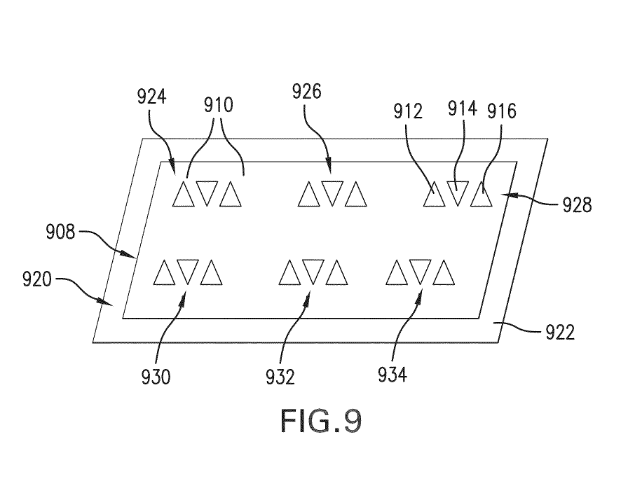

FIG. 9 shows a stencil 902 having openings with triangular edges according to one embodiment of the present invention. Stencil 902 comprising six sets 910 of openings 912, 914 and 916 are formed on a surface 920 of a substrate 922. Each of the six sets 910 of openings has a circular peripheral edge. Although for clarity of illustration, stencil 902 is shown as being substantially two-dimensional in FIG. 9, stencil 902 is actually three-dimensional and has a thickness similar to the thickness of barriers shown in FIGS. 1 and 6. Sets 910 of openings 912, 914 and 916 are at six positions on stencil 902, i.e., position 924, position 926, position 928, position 930, position 932 and position 934. Stencil 902 is suitable for fabricating triangular lipid multilayer structures by following the steps illustrated in FIG. 7.

For simplicity of illustration, in FIG. 9, each set of the six sets of openings has three openings at each of the six 924, 926, 928, 93930, 932, and 934 on substrate 922. However, there may be any number of openings at each of the six positions 924, 926, 928, 930, 932, and 934. There may be any number of positions on a surface of a substrate.

FIGS. 8-9 shows only some embodiments of the edge evaporation lithography (EEL) method for fabricating lipid microarrays using dispersed materials encompassing one or more lipids. The techniques shown in FIGS. 8-9 may be used with other types of dispersed materials to form microarrays of these other types of dispersed materials. For example, barriers may be used to form microarrays of dispersed materials such as: organic liquids, organic solids, gels, liquid crystals, metallic nanoparticles, semiconductor nanoparticles such quantum dots, microscopic beads, etc. In other embodiments, microarray of lipid multilayer structures may be formed for other purposes.

In one embodiment of the present invention, suitable solvents for use in the lipid solutions include ethanol.

The lipid solutions and lipid multilayer structures may each contain one lipid or two or more lipids. In one embodiment of the present invention, the lipid multilayer structures may comprise 1,2-dioleoyl-3-trimethylammoniumpropane (chloride salt) (DOTAP).

The lipid solutions and lipid multilayer microstructures may each contain one drug or two or more drugs.

The method of the present invention for making multiple microarrays of lipid multilayer structures, such as shown in FIGS. 1, 6 and 7, allows for an increase in the number of tests that may be performed on a microplate. For example, the number of tests is increased from being able to test one compound or concentration per well to 6 tests per square centimeter (cm²), which can be used in a standard 24 well cell culture microplate or in any microplate with a larger well area. The total number of tests that can be used in a 24 well microplate is 216 tests (assuming a well diameter of 1.5 cm). This number more than doubles the number of tests that can be performed on the widely used 96 well microplates.

A stencil of the present invention may be formed on a substrate in a variety of ways. For example, a stencil may be formed by replica molding from a master made by photolithography. In this process, the fluid elastomeric precursors are poured over a topographically structured silicon master. A cover can be placed on the silicon to press excess prepolymer out of the way. The prepolymer is cured and the stencil is removed from the mask before being placed onto the substrate. A stencil may also be stamped onto the substrate.

A stencil may be removed from a substrate by carefully peeling the stencil from one edge of the stencil. The stencil can also be peeled parallelly, perpendicularly, horizontally, or at an angle relative to the pattern axis.

Lipid solutions may be added to spaces between barriers by pipetting, or robotic spotting techniques such as pin spotting or inkjet printing ^{37, 38}.

The migration of cells may be measured optically by a microarray scanner, fluorescence or optical microscope, or by the naked eye.

A decrease or increase in the migration of cells beyond lipid multilayer structures bounding the cells indicates an effect of the drug in the lipid multilayer structures on the cells. The distance traveled by the edge of the cells from the starting point provides a measure of cell migration. The effects of different dosages of a drug can also be determined by the effects of lipid microstructures having different dosages of a drug on the migration of cells.

### EXAMPLES

### Materials and Methods

### Chemical Structures

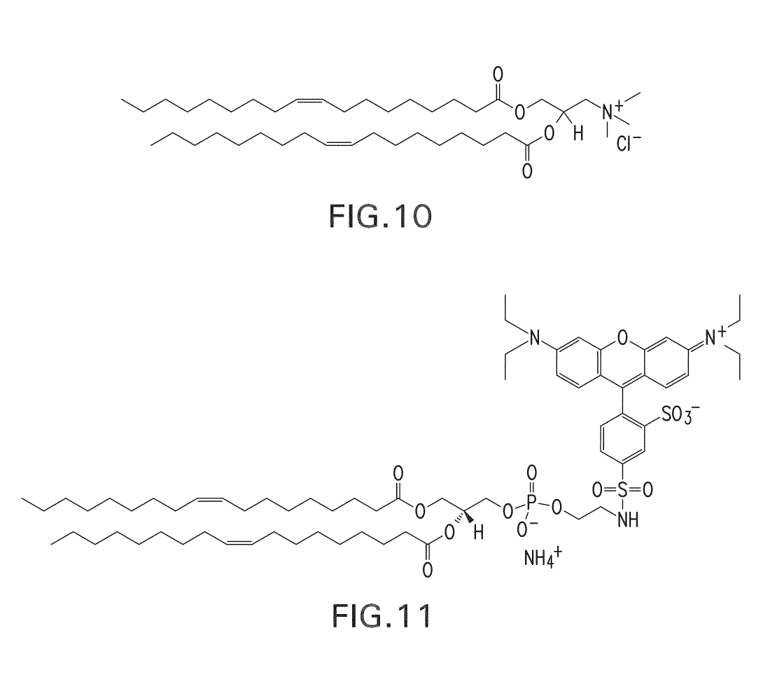

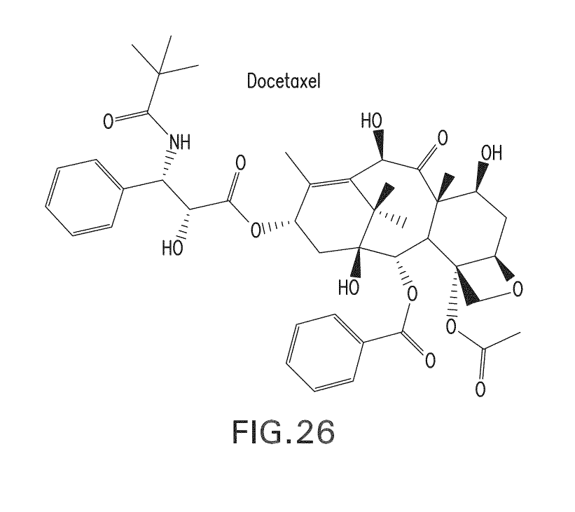

The chemical structures of the lipids and drugs used in various examples are shown in FIGS. 10 and 11. FIG. 10 shows the chemical structure of 1,2-dioleoyl-3-trimethylammonium-propane (chloride salt) (DOTAP). FIG. 11 shows the chemical structure of 1,2-dioleoyl-sn-glycerol-3-phosphoethanolamine-N-(lissamine rhodamine b sulfonyl) (ammonium salt) (DOPE-rhodamine or DOPE-RB). FIG. 26 shows the chemical structure of Taxotere® (docetaxel).

### Preparation of Glass Coverslips for Patterning

Glass coverslips were prepared by cleaning first with detergent (Palmolive soap) and rinsed thoroughly with deionized water. Next the surfaces were subsequently cleaned with rinses in acetone, 100% ethanol, and deionized ultrapure Mill-Q water (EMD Millipore, Massachusetts, USA). Coverslips were dried with a steady stream of nitrogen gas and allowed to completely dry for at least 30 minutes in a biosafety cabinet.

### Edge Evaporation Lithography (EEL)

Polydimethylsiloxane strips (PDMS) was cured from the SYLGARD® 184 silicone elastomer kit in a 60 C oven overnight. PDMS strips of approximately 15 mm long by approximately 1 mm wide were placed between 500 to 800 μm apart on prepared glass coverslips before addition of lipid mixtures. 1,2-dioleoyl-3-trimethylammonium-propane (chloride salt) (DOTAP) and 1,2-dioleoyl-sn-glycerol-3-phosphoethanolamine-N-(lissamine rhodamine b sulfonyl) (ammonium salt) (DOPE-rhodamine) were purchased from Avanti Polar Lipids. To create lipid only solutions, DOTAP was mixed with 1 molar percent (mol %) DOPE-rhodamine in chloroform and dried overnight in a vacuum pump desiccators at 16 LM 15 Torr 7.4 PSIG to remove solvent. Ethanol (100%) was added to suspend dried lipid powder and 1 μl was deposited in each PDMS channel and the array was dried overnight in a vacuum to remove residual ethanol. A similar procedure was used for docetaxel encapsulated lipid solutions and the drug was dissolved in ethanol prior to adding to the dried lipid powder.

### General Cell Culture and Staining

HeLa cells (obtained from the American Type Culture Collection and maintained according to the collections guidelines) for all experiments were seeded at 2.5×10⁵ cells/ml and grown to approximately 70% confluence in growth media composed of Dulbecco's Modified Eagle Medium supplemented with 10% Cosmic Calf Serum. Cells were incubated at 37° C. and 5% CO₂. Trypsin with EDTA (0.25%) was used for cell detachment and the medium was replaced with fresh growth medium 24 hours before the experiment. Prior to imaging, cells were stained for viability 20 minutes with live-cell fluorescent dye (SYTO9) and propidium iodide in Hank's buffered saline solution.

### Viability Assays

Cell viability was determined using the BacLight viability assay from Invitrogen®.

### Cell Adhesion Experiments

Lipids were dissolved in ethanol and added to each channel in increasing amounts from 0.2 ng to 20 μg. Tests for cell attachment were performed by seeding HeLa cells into an assay channel (a channel between two PDMS strips) with varying amounts of lipid films. Cells were allowed to attach for two hours before washing the channels repeatedly 5 times. Prior to counting, cells were incubated with live-cell fluorescent dye (SYTO9). The number of cells per square micron area (μm²) that remained attached was counted manually. Experiments for cell adhesion where performed in three replicates. Values for cell density of each treatment were determined by averaging the number of cells in 5 random 100 μm² areas in a single image captured at a 4× magnification.

### Characterization of Lipid Films

The free spaces in between PDMS stencils of the assay that contained lipid material were imaged with a G-2E/C red fluorescence filter. Characterization of the lipid films was performed by averaging the maximum intensity of 10 random cross sections on both edges of the barrier for a total of 20 samples using the freeware analysis software, ImageJ®. The middle of the channels was determined by 10 random cross sectional areas.

### Cell Migration Assay of HeLa Cells

The patterned glass coverslips were placed individually into each well of a 6-well plate for cell culture. For experiments, cells were seeded onto the prepared patterned glass coverslips by gently pipetting the cell suspension (2.5×10⁵ cells/ml, 1 ml per coverslip) directly over each channel between each pair of PDMS barriers to allow cells to settle in them. The same method was used to seed cells onto other parts of the coverslips for use as control areas. Cells were allowed to settle for 1-2 hours before the PDMS barriers were removed to promote cell migration. After PDMS barriers being removed, the coverslips were washed once with HBSS and replaced with fresh growth media. The cells were incubated over the patterned areas for about 24 hours. The width of each array of cell cultures was manually measured with Nikon Elements 4.0 analysis software and the average migration rate for each strip was determined using Equation 1 below:

$$Migration Rate ( μ m \text{/} hr ) = \begin{matrix}{Width}_{T = 24 hrs} - {Width}_{T = 0 hrs} \\ 24 hrs\end{matrix} \qquad ( 1 )$$

Width is the average width in micrometers (μm) of the cell monolayer measured either at the beginning of the experiment (T=0 hrs) or at the end (T=24 hours) of the experiment.

### Microscopy

The images for cell migration were captured on a Nikon Ti Eclipse inverted microscope with 4× or 10× objectives. Images for cell migration were taken at 0 hour after barrier removal and once again 24 hours after incubation period in phase contrast. Fluorescent filters used were B-2E/C and G-2E/C for red and green emitting dyes.

### Statistical Analysis

A Student's t-test was performed to determine the statistical significance between means of each sample (p-value <0.05). Error bars in figures are standard error of the mean for n samples. Characterizing the edge lipid films produced a total of n=20 samples and middle lipid film measurements produced n=10 samples. Cell density experiments were performed in triplicates (n=3). All samples for cell migration were taken in triplicate and replicated three times (n=9).

### Example 1

### Characterization of Lipid Films for Cell Migration Assay

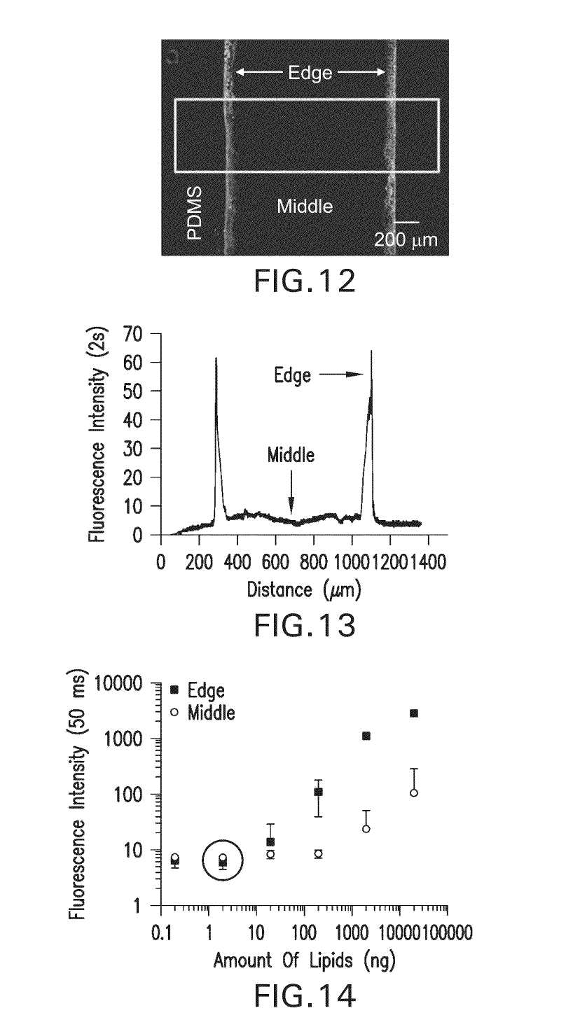

The optimal thickness of lipid films was initially determined using fluorescent analysis shown in FIGS. 12, 13, and 14. FIG. 12 shows a fluorescence image of lipid multiplayer array at starting lipid concentration of 2 μg/ml (selection in white used for intensity profile). FIG. 13 shows a plot profile graph of the vertically averaged fluorescence intensity profile across the horizontal cross section outlined in FIGS. 12 and 14. Previously, it has been shown that fluorescent intensity of DOPE-rhodamine doped lipids is directly correlated to lipid multilayer height or thickness.¹⁸ A proportional relationship was also observed from these lipid films between sensitivity (fluorescence intensity versus camera exposure time) and the amount of lipids added between the PDMS stencils. The thickness of the lipid multilayers can be controlled by the amount of lipids added between the stencils (FIG. 14). The fluorescence data for the edge and middle were taken at different exposure times for imagine. The circled point in FIG. 14 indicates the concentration used in FIGS. 12 and 13. and data is expressed as standard error of the mean. The initial concentration of lipid determined how the lipids dried within the PDMS channels. Adding lipid at concentration of 200 μg/ml (i.e., amount of lipid is 200 ng) or higher caused excess lipids to be dried within the middle of the PDMS stencils in addition to thicker multilayers on the edges of the stencil. However, lower amounts of lipid only dried to the edges of the barriers (FIG. 14). This control of multilayer thickness by concentration was important when cells were added to the assay.

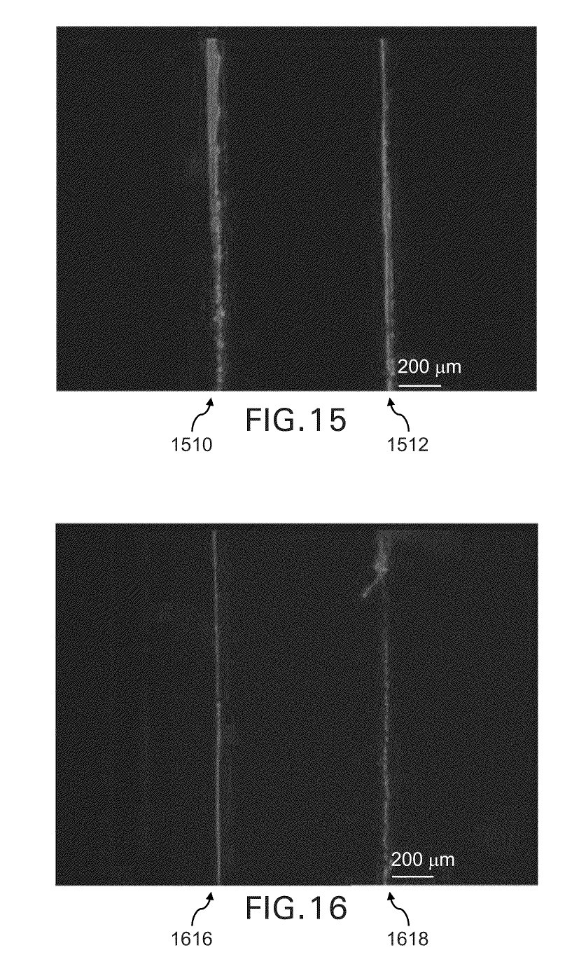

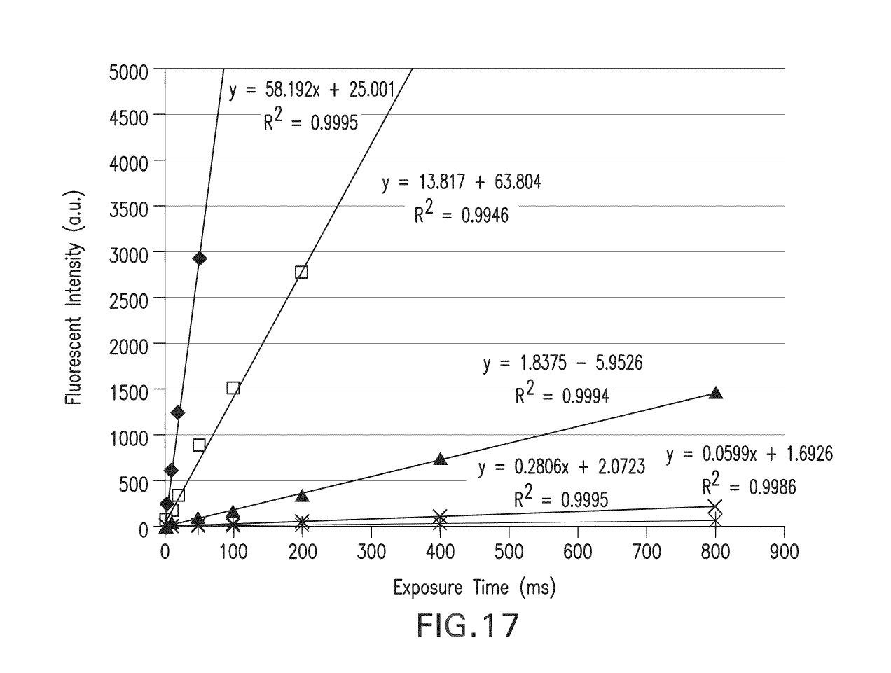

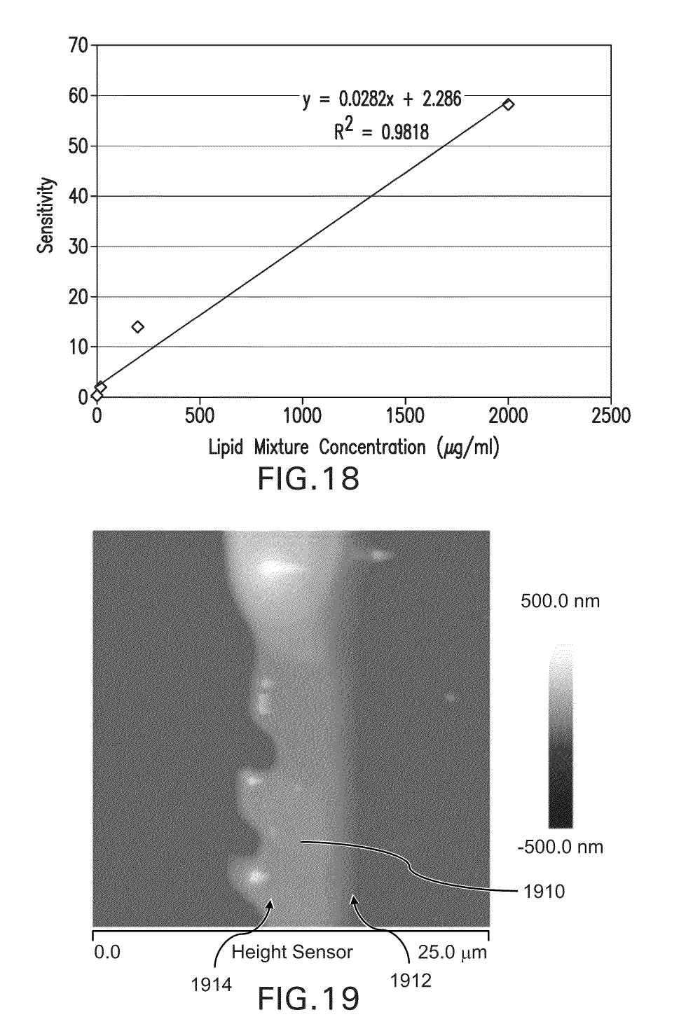

The fluorescence images of arrays at different lipid concentrations are further shown in FIGS. 15 and 16. FIG. 15 shows the lipid film dried from lipid solution at the concentration of 200 μg/ml. FIG. 16 shows the lipid film dried from lipid solution at the concentration of 20 μg/ml. FIG. 17 shows the graph of fluorescence intensity of lipid patterns at increasing exposure times (slop is sensitivity). FIG. 18 shows the graph sensitivity as a function of concentration.

FIG. 19 is an atomic force microscopy of lipid array 1910 formed on a substrate from lipid solution at concentration of 200 μg/ml after removal of barriers. A linear edge 1912 of a lipid array 1910 is formed at the side directly contacting an edge of a barrier. Non-linear edge 1914 of lipid array 1910 is formed at the side closer to the center of the space bounded by a pair of barriers.

### Example 2

### Cell Adhesion on Different Lipid Multilayer Thicknesses

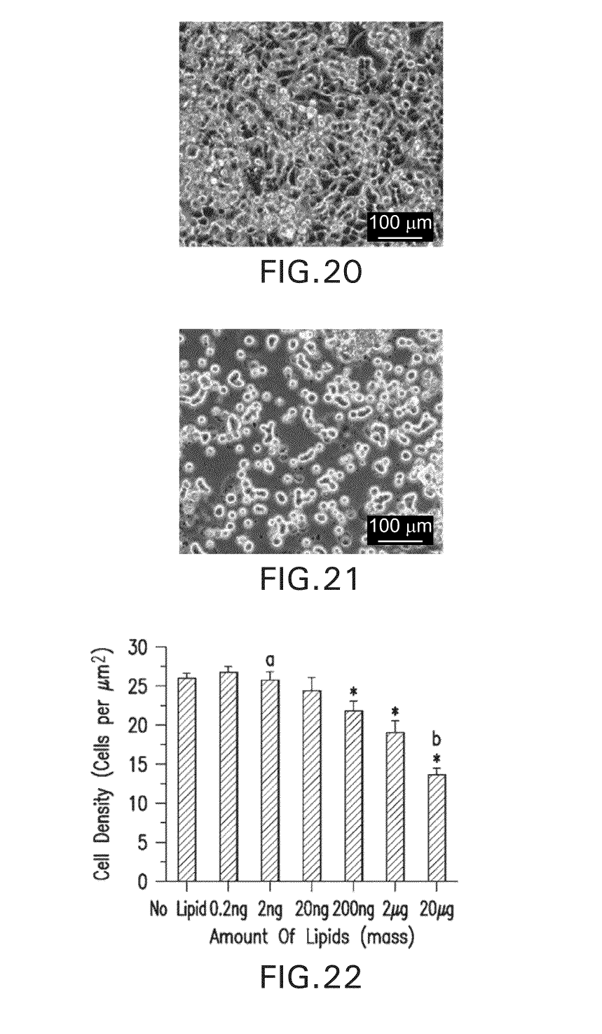

The effect of lipid multilayer thickness on cell adhesion was determined in FIGS. 20, 21, and 22. HeLa cells grown on lipid multilayers created from 200 ng (initial solution with concentration of 200 μg/ml) of lipids or more exhibited abnormal morphology and appeared dead compared to thinner multilayers (FIGS. 20 and 21). As shown in FIG. 20, HeLa cells spread out and attached in channels on the substrate created from lipid with concentration of 2 μg/ml in initial solution. In FIG. 21, HeLa cells poorly spread out and attached on the substrate created from lipid with concentration of 20 mg/ml in initial solution. A higher multilayer thickness results in cells looking balled and not spread out leading to apoptosis (FIG. 21). This toxic effect could be from the cationic lipid DOTAP that was used, which can be cytotoxic at high concentrations.⁷⁸

FIG. 22 is a graph of adherent cell density versus lipid solution concentration used to form films in assay channels (concentrations used in FIG. 20 and FIG. 21 are indicated by letter a and b, respectively.) As shown in FIG. 22, cells were adherent to lipid films created from 20 ng (initial solution with concentration of 20 μg/ml) or lower but began to adhere significantly less (p<0.05) at higher concentrations compared to untreated glass (FIG. 20). The asterisks represent significant different from control (p<0.05). Images and data were collected after 2 hours of incubation and data is expressed as standard error of the mean.

These findings corroborate other studies that cells have poor adhesion to certain material surfaces such as lipid bilayers.⁷⁷ High concentrations of lipid on the surface could also influence the ability of cells to attach to the surface normally by disrupting the ability of adhesion proteins to interact with the surface. Additionally, higher numbers of dead cells were observed over regions with larger amounts of lipid (data not shown).

### Example 3

### Cell Migration on Different Lipid Multilayer Thicknesses

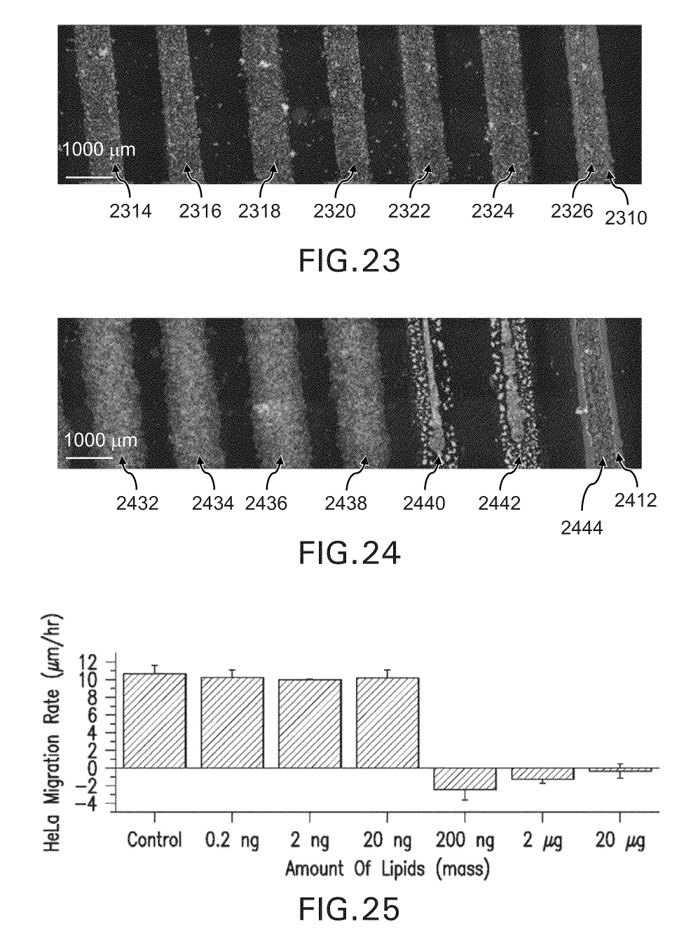

The effect of lipid multilayer thickness on HeLa cell migration was determined in FIGS. 23, 24, and. Phase contrast and fluorescent micrographs were taken immediately after PDMS barrier removal and 24 hours after to measure the migration rate over time. To visualize the amount of lipid on the surface, rhodamine-DOPE lipid was doped at 1 molar percent with the DOTAP lipid. The cells were stained with SYTO9, a live cell DNA stain.

Initial HeLa cell epithelial sheets before migration are shown in FIG. 23 and HeLa cell epithelial sheets 24 hours after migration are shown in FIG. 24. Lipid spots are shown in red indicated by arrows 2310 and 2412. Each cell strips or arrays of cell cultures 2314, 2316, 2318, 2320, 2322, 2324, and 2326 was formed on lipid films with lipid at the amount of 0, 0.2 ng, 2 ng, 20 ng, 200 ng, 2 μg, and 20 μg, respectively (i.e., lipid films made from initial lipid solutions at the concentration of 0 ng/ml, 200 ng/ml, 2 μg/ml, 20 μg/ml, 200 μg/ml, 2 mg/ml, and 20 mg/ml) (left to right). Each Cell strips 2432, 2434, 2436, 2438, 2440, 2442, and 2444 in FIG. 24 each shows cell migration 24 hours after contacting with the lipid films at the amount of 0, 0.2 ng, 2 ng, 20 ng, 200 ng, 2 μg, and 20 μg, respectively. Images in FIGS. 23 and 24 are 6×3 stitched micrograph images captured with a motorized stage. The asterisk represents significant difference from control (p<0.05) and data is expressed as standard error of the mean. Scale bars are 1000 μm.

As shown in FIG. 25, the migration rate of HeLa cells was not significantly (p<0.05) affected by lipid thickness in channels created from lipid solutions at a concentration of 20 μg/ml or lower but were significantly hindered at higher concentrations. Excess lipid on the surface causes either reduced cell attachment or cell death which significantly reduces the ability of the cell strip to migrate. Therefore, 20 ng of lipid (lipid solution at a concentration of 20 μg/ml in ethanol) was used for all migration assays.

### Example 5

### Effect of Docetaxel from Lipid Multilayer Films on Cell Migration

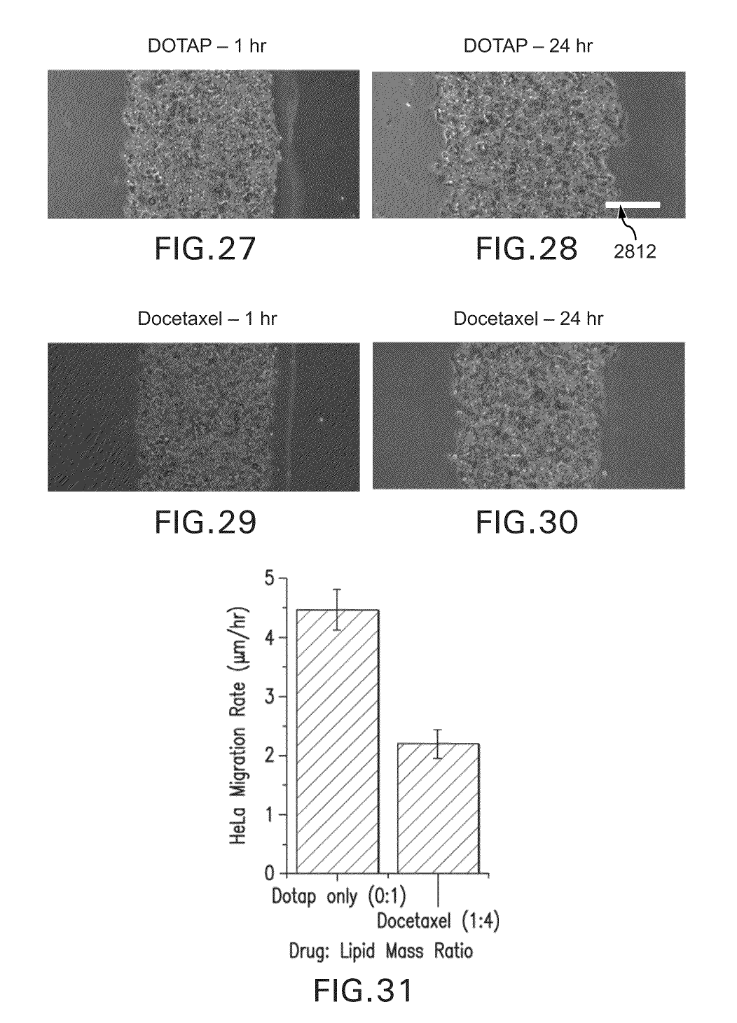

FIGS. 27, 28, 29, 30 and 31 show a lipid-based cell assay to investigate the effect of the antimicrotubule drug docetaxel that inhibits intracellular protein transport on HeLa cellular migration rate. The migration assay was tested with a lipophilic drugs docetaxel, which is poorly soluble in water (less than 0.025 mg/L) and has a log P of 4.1.^{79,80} The ratio of docetaxel to lipid ratios (by mass) in this assay is 1:4. Docetaxel were delivered into HeLa cells by uptake from encapsulated lipid films.

FIG. 27 shows a merged micrograph of HeLa cell strip (in phase contrast) in contact with a DOTAP only fluorescent lipid film (doped with 1 mol % DOPE-rhodamine), 1 hour after PDMS barriers were removed. FIG. 28 shows the HeLa cell strip of FIG. 27, 24 hours after the PDMS barriers were removed. FIG. 29 is a merged image of a HeLa strip incubated with a docetaxel-encapsulated fluorescent lipid film, 1 hour after PDMS barriers were removed. FIG. 30 shows the HeLa cell strip of FIG. 29, 24 hours after the PDMS barriers were removed. A scale bar 2812 is shown in and is 200 μm.

A graph of migration rate (in μm/hr) versus drug to lipid mass ration in FIG. 31 shows a significantly (p<0.05) reduced collective cell migration compared to DOTAP only control. Data for each treatment was performed in triplicate twice (n=6) and is expressed as standard error of the mean.

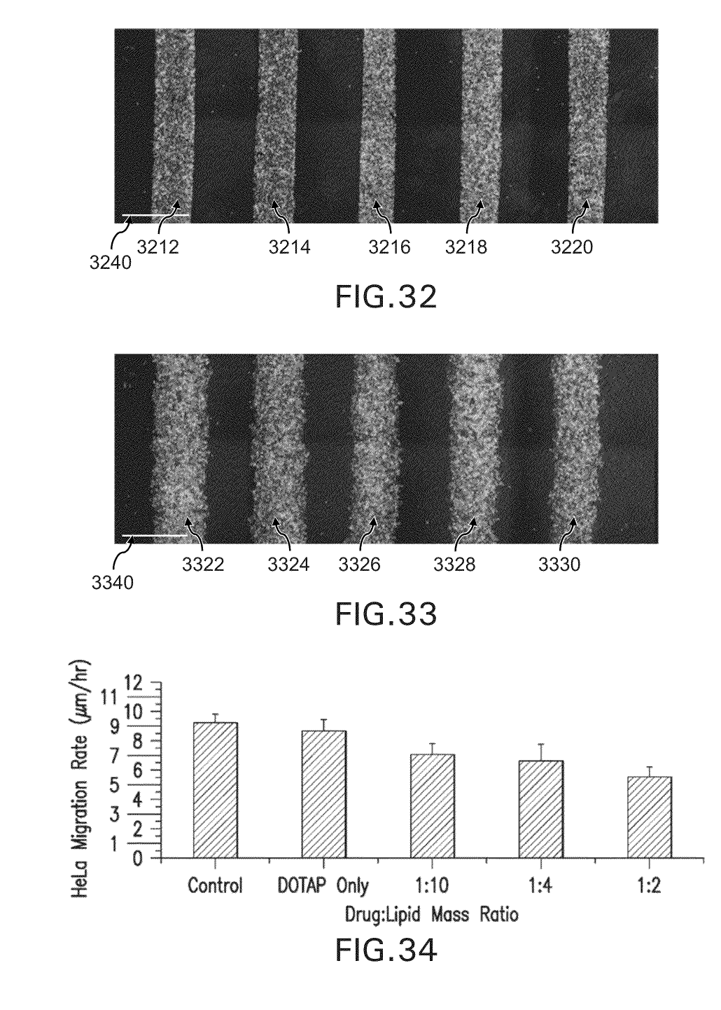

Additionally, three different docetaxel to lipid ratios (by mass) of 1:10, 1:4 and 1:2 were able to be assayed at once on the same array, as shown in FIGS. 32, 33, and 34. FIGS. 32 and 33 show 4×3 stitched 4× images captured with a motorized stage.

FIG. 32 shows each HeLa cell strips at time 0 hour after contact with lipid encapsulated docetaxel films. Channel 3212 is a lipid free control. Channel 3214 has a DOTAP only fluorescent lipid film (doped with 1 mol % DOPE-rhodamine). Lipid films in channel 3216, 3218, and 3200 were formed at docetaxel to lipid ratios (by mass) of 1:10, 1:4 and 1:2, respectively. FIG. 33 shows each HeLa cell strips of FIG. 32, 24 hours after the PDMS barriers were removed, wherein channel 3322 corresponding to channel 3212, channel 3324 corresponding to channel 3214, channel 3326 corresponding to channel 3216, channel 3328 corresponding to channel 3218, and channel 3330 corresponding to channel 3220, respectively. In FIGS. 32 and 33, lipids were stained with DOPE-rhodamine and cells were stained with SYTO9 DNA stain (green). In FIG. 33, propidium iodide stain (red) was further used to show cell viability after 24 hours. A scale bar 3240 in FIG. 32 and a scale bar 3340 in FIG. 33 are each 1000 μm.

A graph of migration rate (in μm/hr) versus drug to lipid mass ration in FIG. 34 shows a significantly (p<0.05) reduced collective cell migration compared to control group. These results indicated that docetaxel delivered by encapsulated lipid films reduced the migration rate of the cells dose-dependently over 24 hours due to disruption of microtubule dynamics by docetaxel. In FIG. 34, data is expressed as standard error of the mean.

Docetaxel is most likely influencing many different cell processes such as inhibiting cell division and migration while increased apoptosis. These effects in combination can reduce the ability of a monolayer of cells to migrate across the glass coverslip compared to untreated cells. Therefore, it is believed that docetaxel should have some inhibitory effect on the ability of HeLa cells to migrate in a coordinated fashion.

As shown in FIGS. 27, 28, 29, 30, 31, 32, 33 and 34, HeLa cell migration is inhibited by docetaxel, which suggests that docetaxel and other taxol derivatives could be used to target different cell processes for cancer therapies.

One advantage of this lipid-based surface delivery system over other existing assays is that it allows the study of the effects of poorly water soluble compounds such as docetaxel on cell movement following drug delivery into the cells. Different amounts or types of compounds can also be tested at the same time in parallel which leads to a reduced amount time for running separate tests. Another advantage is that uptake of drugs into cells can be facilitated without DMSO, which can be hazardous to work with because it functions as a chemical carrier and easily penetrates the skin along with solubilized compounds. Additionally, this assay requires smaller amounts of drug per assay as compared to a standard scratch migration assay which requires dissolving the drug at certain concentrations in each micro-well. Furthermore, migrating cells on the edge of the barrier region are exposed locally to lipid encapsulated drug compared to proliferating cells in the interior region of monolayer culture.

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

### Example 6

### Edge Evaporation Lithography (EEL) on Three Different Chemically Treated Surfaces Using Hexadecane

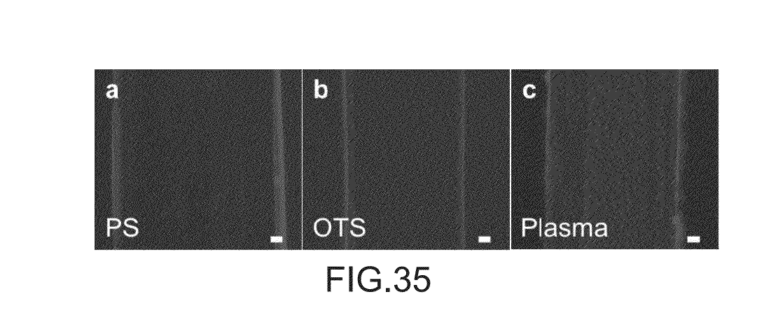

FIG. 35 demonstrates edge evaporation lithography (EEL) on three different chemically treated surfaces using hexadecane. Hexadecane is first dissolved in ethanol to desired concentration of 20 mg/L. The dye TRITC (tetramethyl-rhodamine isothiocyanate) is dissolved in the ethanol at 1 molar percent of the hexadecane amount to dope the film once ethanol is evaporated. Panels a-c are fluorescence images capturing TRITC and hexadecane film locations within PDMS barriers after solvent evaporation seen in method 102 of FIG. 1. Panel a shows that a hexadecane film forms lines along PDMS barrier edges. The film in Panel a is about 50 micrometers wide on an untreated polystyrene (PS) surface. Panel b shows that a hexadecane film forms lines along the edges of PDMS barriers on a hydrophobic OTS (triethoxycaprylylsilane) treated glass coverslip. The film width is less than 50 micrometers and the film is much thinner on OTS than that on PS or Plasma. In panel c, the hexadecane film covers the whole width of the channel on a hydrophilic plasma treated glass coverslip (plasma), which is in contrast to the edge film created from PS and OTS. Hexadecane films created by EEL can be controlled by the surface treatment. The scale in all panels represents 50 micrometers.

### REFERENCES

The following references are referred to above and are incorporated herein by reference:
1. Barenholz, Y., Gibbes, D., Litman, B. J., Goll, J., Thompson, T. E., and Carlson, F. D., “A simple method for the preparation of homogeneous phospholipid vesicles,” Biochemistry 16, 2806-10 (1977).
2. Szokam F., and Papahadjopoulos, D., “Comparative properties and methods of preparation of lipid vesicles (liposomes),” Annu. Rev. Biophys. Bio 9, 467-508 (1980).
3. Gustafsson, J., Arvidson, G., Karlsson, G., and Almgren, M. “Complexes between cationic liposomes and DNA visualized by Cryo-Tem,” BBA-Biomembranes 1235, 305-12 (1995).
4. Kwon, C. H., Wheeldon, I., Kachouie, N. N., Lee, S. H., Bae, H., Sant, S., Fukuda, J., Kang, J. W., Khademhosseini, A., “Drug-eluting microarrays for cell-based screening of chemical-induced apoptosis,” Anal. Chem. 83, 4118-25 (2011).
5. Malam, Y., Loizidou, M., and Seifalian, A. M., “Liposomes and nanoparticles: nanosized vehicles for drug delivery in cancer,” Trends Pharmacol. Sci. 30, 592-99 (2009).
6. Porter, C. J. H., Trevaskis, N. L., and Charman, W. N., “Lipids and lipid-based formulations:optimizing the oral delivery of lipophilic drugs,” Nat. Rev. Drug Discov. 6, 231-48 (2007).
7. Torchilin, V. P., “Micellar nanocarriers: pharmaceutical perspectives,” Pharm. Res. 24, 1-16 (2007).
8. Koren, E., and Torchilin, V. P., “Drug carriers for vascular drug delivery,” IUBMB Life 63, 586-95 (2011).
9. Gregoriadis, G., “Engineering liposomes for drug delivery: progress and problems,” Trends in Biotechnology 13, 527-37 (1995).
10. Kusi-Appiah, A. E., Vafai, N., Cranfill, P. J., Davidson, M. W., and Lenhert, S., “Lipid multilayer microarrays for in vitro lipomosomal drug delivery and screening,” Biomaterials 33, 4187-94 (2012).
11. Majd, S, and Mayer, M., “Hydrogel stamping of arrays of supported lipid bilayers with various lipid compositions for the screening of drug-membrane and protein-membrane interactions,” Angew. Chem. Int. Ed. 44, 6697-6700 (2005).
12. Moran-Mirabal, J. M., Edel, J. B., Meyer, G. D., Throckmorton, D., Singh, A. K., and Craighead, H. G., “Micrometer-sized supported lipid bilayer arrays for bacterial toxin binding studies through total internal reflection fluorescence microscopy,” Biophys. J. 89, 296-305 (2005).
13. Deng, Y., Wang, Y., Holtz, B. Li, J., Traaseth, N., Veglia, G., Stottrup, B. J., Elde, R., Pei, D., Guo, A., and Zhu, X. Y., “Fluidic and air-stavle supported lipid bilayer and cell-mimicking microarrays,” J. Am. Chem. Soc. 130, 6267-71 (2008).
14. Yamazaki, V., Sirenko, O., Schafer, R. J., Nguyen, L., Gutsmann, T., Brade, L., and Groves, J. T., “Cell membrane array fabrication and assay technology,” BMC Biotechnology 2005, doi:10.1186/1472-6750-5-18 (2005).
15. Lenhert, S., Brinkmann, F., Laue, T., Walheim, S., Vannahme, C., Klinkhammer, S., Xu, M., Sekula, S., Mappes, T., Schimmel, T., and Fuchs, H., “Lipid multilayer gratings,” Nat. Nanotechnol. 5, 275-79 (2010).
16. Lenhert, S., Sun, P., Wang, Y. H., Fuchs, H., and Mirkin, C. A., “Massively parallel dip-pen nanolithography of heterogeneous supported phospholipid multilayer patterns,” Small 3, 71-75 (2007).
17. Sekula, S., Fuchs, J., Weg-Remers, S., Nagel, P., Schuppler, S., Fragala, J., Theilacker, N., Franzreb, M., Wingren, C., Ellmark, P., Borrebaeck, C. A. K., Mirkin, C. A., Fuchs, H., and Lenhert, S., “Multiplexed lipid dip-pen nanolithography on subcellular scales for the templating of functional proteins and cell culture,” Small 4, 1785-93 (2008).
18. Nafday, O. A., and Lenhert, S. “High-throughput optical quality control of lipid multilayers fabricated by dip-pen nanolithography,” Nanotechnology 22, doi:225301 (2011).
19. Perino-Gallice, L., Fragneto, G., Mennicker, U., Salditt, T., and Rieutord, F., “Dewetting of solid-supported multilamellar lipid layers,” Eur. Phys. J. E 8, 275-82 (2002).
20. Mathieu, M. Schunk, D., Franzka, S., Mayer, C., and Hartmann, N., “Temporal stability of photothermally fabricated micropatterns in supported phospholipid multilayers,” J. Vac. Sci. Technol. A 28, 953-57 (2010).
21. Perl, A., Reinhoudt, D. N., and Huskens, J., “Microcontact printing: limitations and achievements,” Adv. Mater. 21, 2257-68 (2009).
22. Nafday, 0. A., Lowry, T. W., and Lenhert, S., “Multifunctional lipid multilayer stamping,” Small 8, 1021-28 (2012).
23. Heller, M. J., “DNA microarray technology: devices, systems, and applications,” Annu. Rev. Biomed. Eng. 4, 129-53 (2002).
24. Howbrook, D. N., van der Valk, A. M., O'Shaughnessy, M. C., Sarker, D. K., Baker, S.C., and Lloyd, A. W., “Developments in microarray technologies,” Drug Discov. Today 15, 648-51 (2003).
25. Eteshola, E., and Leckband, D., “Development and characterization of an ELISA assay in PDMS microfluidic channels,” Sens. Actuator B-Chem. 72, 129-33 (2001).
26. Braunschweig, A. B., Huo, F. W., and Mirkin, C. A., “Molecular printing,” Nat. Chem. 1, 353-58 (2009).
27. Salaita, K., Wang, Y. H., and Mirkin, C. A., “Applications of dip-pen nanolithography,” Nat. Nanotechnol. 2, 145-55 (2007).
28. Ginger, D. S., Zhang, H., and Mirkin, C. A., “The evolution of dip-pen nanolithography,” Angew. Chem. Int. Ed. 43, 30-45 (2004).
29. Piner, R. D., Zhu, J., Xu, F., Hong, S. H., and Mirkin, C. A., “Dip-pen” nanolithography,” Science 283, 661-63
30. Salaita, K., Wang, Y. H., Fragala, J., Vega, R. A., Liu, C., Mirkin, C. A. “Massively parallel dip-pen nanolithography with 55000-pen two-dimensional arrays,” Angew. Chem. Int. Ed. 45, 7220-23 (2006).
31. Zhang, M., Bullen, D., Chung, S. W., Hong, S., Ryu, K. S., Fanm Z. F., and Mirkin, C. A., and Liu, C., “A MEMS nanoplotter with high density parallel dip-pen nanolithography probe arrays,” Nanotechnology 13, 212-17 (2002).
32. Xia, Y. N., and Whitesides, G. M., “Soft lithography,” Annu. Rev. Mater. Sci. 28, 153-84 (1998).
33. Huo, F., Zheng, Z, Zheng, G, Giam, L., Zhang, H., and Mirkin, C. A., “Polymer pen lithography,” Science 321 1658-60 (2008).
34. Kusi-Appiah, A., Vafai, N., Cranfill, P. J., Davidson, M. W. & Lenhert, S., “Lipid multilayer microarrays for in vitro liposomal drug delivery and screening,” Biomaterials 33(16) 4187-94 (2012).
35. Jong, J. W., Smetana, A., and Stiles, P., “Multi-ink pattern generation by dip-pen nanolithography,” Scanning 32, 24-29 (2010).
36. Chou, S. Y., Krauss, P. R., and Renstrom, P. J., “Imprint lithography with 25-nanometer resolution,” Science 272, 85-87 (1996).
37. Torchilin, V. P., “Recent advances with liposomes as pharmaceutical carriers,” Nat. Rev. Drug Discov. 4, 145-60 (2005).
38. Lenhert, S., Mirkin C. A., and Fuchs, H., “In situ lipid dip-pen nanolithography under water,” Scanning 32, 15-23 (2010).
39. Mendez-Vilas, A., Jodar-Reyes, A. B., and Gonzalez-Martin, M. L., “Ultrasmall liquid droplets on solid surfaces: production, imaging, and relevance for current wetting research,” Small 5, 1366-90 (2009).
40. Szymanski, P., Markowicz, M. and Mikiciuk-Olasik, E. “Adaptation of High-Throughput Screening in Drug Discovery-Toxicological Screening Tests,” International Journal of Molecular Sciences 13, 427-452 (2012).
41. Sampieri, K. and Fodde, R., “Cancer stem cells and metastasis,” Seminars in Cancer Biology., 22, 187-193 (2012).
42. Brabletz, T., Jung, A., Spaderna, S., Hlubek F., and Kirchner, T., “Opinion: migrating cancer stem cells—an integrated concept of malignant tumour progression.” Nature Reviews Cancer, 5, 744-749 (2005).
43. Eilken, H. M. and Adams, R. H., “Dynamics of endothelial cell behavior in sprouting angiogenesis,” Current Opinion in Cell Biology, 22, 617-625 (2010).
44. Griffioen, A. W. and Molema, G., “Anti-angiogenesis: making the tumor vulnerable to the immune system,” Pharmacology Review., 52, 237-268 (2000).
45. Witte, M. B. and Barbul, A., “General principles of wound healing,” The Surgical Clinics of North America., 77, 509-+(1997).
46. Aman, A. and Piotrowski, T. “Cell migration during morphogenesis,” Developmental Biology, 341, 20-33 (2010).
47. Weijer, C. J. “Collective cell migration in development,” Journal of Cell Science., 122, 3215-3223 (2009).
48. Liang, C. C., Park, A. Y. and Guan, J. L., “In vitro scratch assay: a convenient and inexpensive method for analysis of cell imgration invitro,” Nature Protocols., 2, 329-333 (2007).
49. Valster, A., Tran, N. L., Nakada, M., Berens, M. E., Chan A. Y. and Symons, M., “Cell migration and invasion assays,” Methods, 37, 208-215 (2005).
50. van Horssen, R. and ten Hagen, T. L. M., “Crossing barriers: The new dimension of 2D cell migration assays,” Journal of Cellular Physiology., 226, 288-290 (2011).
51. Lenhert, S., Meier, M. B., Meyer, U., Chi, L. F. and Wiesmann, H. P., “Osteoblast alignment, elongation and migration on grooved polystyrene surfaces patterned by Langmuir-Blodgett lithography,” Biomaterials, 26, 563-570 (2005).
52. Shin, K. D., Lee, M. Y., Shin, D. S., Lee, S., Son, K. H., Koh, S. Paik, Y. K., Kwon B. M. and Han, D.C., “Blocking tumor cell migration and invasion with biphenyl isoxazole derivative KRIBB3, a synthetic molecule that inhibits Hsp27 phosphorylation,” Journal of Biological Chemistry., 280, 41439-41448 (2005).
53. Gough, W., Hulkower, K. I., Lynch, R., McGlynn, P., Uhlik, M., Yan, L. and Lee, J. A, “A quantitative, facile, and high-throughput image-based cell migration method is a robust alternative to the scratch assay,” Journal of Biomolecular Screening, 16, 155-163 (2011).
54. Attoub, S., Hassan, A. H., Vanhoecke, B., Iratni, R., Takahashi, T. Gaben, A.-M., Bracke, M., Awad, S., John, A., Kamalboor, H. A., Al Sultan, M. A. Arafat, K. Gespach, C. and Petroianu, G., “Inhibition of cell survival, invasion, tumor growth and histone deactylase activity by the dietary flavonoid luteolin in human epitholioid cancer cells,” European Journal of Pharmacology, 651, 18-25 (2011).
55. Chung, S., Sudo, R., Mack, P. J., Wan, C. R., Vickerman, V. and Kamm, R. D., “Cell migration into scaffolds under co-culture conditions in a microfluidic platform,” Lab on a Chip, 9, 269-275 (2009).
56. Conant, C. G., Nevill, J. T., Schwartz, M. and Ionescu-Zanetti, C., “Wound healing assays in well-plate coupled microfluidic devices with controlled parallel flow,” Journal of the Association for Laboratory Automation, 15, 52-57 (2010).
57. Huang, X. W., Li, L., Tu, Q., Wang, J. C., Liu, W. M., Wang, X. Q., Ren, L. and Wang, J. Y., “On-chip cell migration assay for quantifying the effect of ethanol on MCF7 human breast cancer cells,” Microfluid Nanofluid, 10, 1333-1341 (2011).
58. Poujade, M., Grasland-Mongrain, E., Hertzog, A, Jouanneau, J., Chavrier, P., Ladoux, B., Buguin, A. and Silberzan, P. “Collective migration of an epithelial monolayer in response to a model wound,” Proceedings of the National Academy of Sciences of the United States of America, 104, 15988-15993 (2007).
59. Wang, L., Zhu, J., Deng, C., Xing, W. L. and Cheng, J., “An automatic and quantitative on-chip cell migration assay using self-assembled monolayers combined with real-time cellular impedance sensing,” Lab on a Chip, 8, 872-878 (2008).
60. Kim, B. J. and Wu, M. M., “Microfluidics for mammalian cell chemotaxis,” Annals of Biomedical Engineering, 40, 1316-1327 (2012).
61. Liu, T. J., Lin, B. C. and Qin, J. H., “Carcinoma-associated fibroblasts promoted tumor spheroid invasion on a microfluidic 3D co-culture device,” Lab on a Chip, 2010, 10, 1671-1677.
62. Wang, Z., Kim, M.-C., Marquez, M. and Thorsen, T., “High-density microfluidic arrays for cell cytotoxicvity analysis,” Lab on a Chip, 7, 740-745 (2007).
63. Kwak, Y. H., Hong, S. M. and Park, S. S., “A single cell tracking system in real-time,” Cellular Immunology, 265, 44-49 (2010).
64. Puliafito, A., Hufnagel, L., Neveu, P., Streichan, S., Sigal, A., Fygenson, D. K. and Shraiman, B. I. “Collective and single cell behavior in epithelial contact inhibition,” Proceedings of the National Academy Sciences of the United States of America, 109, 739-744 (2012).
65. Adanja, I., Megalizzi, V., Debeir, O., and Decaestecker, C. “A new method to address unmet needs for extracting individual cell migration features from a large number of cells embedded in 3D volumes,” PLoS One, 6 (2011).
66. Diaz-Mochon, J. J., Tourniaire, G. and Bradley, M., “Microarray platforms for enzymatic and cell-based assays,” Chemical Society Reviews, 36, 449-457 (2007).
67. Yarrow, J. C., Totsukawa, G., Charras, G. T. and Mitchison, T. J. “Screening for cell migration inhibitors via automated microscopy reveals a Rho-kinase inhibitor,” Chemistry & Biology, 12, 385-395 (2005).
68. Bailey, S, N., Sabatini D. M. and Stockwell, B. R., “Microarrays of small molecules embedded in biodegradable polymers for use in mammalian cell-based screens,” Proceedings of the National Academy Sciences of the United States of America, 101, 16144-16149 (2004).
69. Tourniaire, G., Collins, J., Campbell, S., Mizomoto, H. Ogawa, S., Thaburet, J. F. and Bradley, M. “Polymer microarrays for cellular adhesion,” Chemical Communications, 2118-2120 (2006).
70. Balakin, K. V., Savchuk, N. P. and Tetko, I. V., “Inh silico approaches of aqueous and DMSO solubility of drug-like compounds: trends, problems and solutions,” Current Medicinal Chemistry, 13, 223-241 (2006).
71. Jacob, S. W. and Herschler, R. “Pharmacology of DMSO,” Cryobiology, 23, 14-27 (1986).
72. Grein, T. A., Freimark, D., Weber, C., Hudel, K., Wallrapp, C. and Czermak, P., “Alternatives to dimethylsulfoxide for serum-free cryopreservation of human mesenchymal stem cells,” International Journal of Artificial Organs, 2010, 33, 370-380.
73. Majd, S. and Mayer, M., “Generating arrays with high content and minimal consumption of functional membrane proteins,” Journal of American Chemical Society, 130, 16060-16064 (2008).
74. Diguet, A., Le Berre, M., Chen, Y. and Baigl, D., “Preparation of phospholipid multilayer patterns of controlled size and thickness by capillary assembly on a microstructured substrate,” Small, 5, 1661-1666 (2009).
75. Brinker, C. J., Lu, Y. F., Sellinger, A. and Fan, H. Y., “Evaporation-induced self-assembly: Nanostructures made easy,” Advanced Materials, 11, 579-+(1999).
76. Yuan, B., Xing, L. L., Zhang, Y. D., Lu, Y., Mai, Z. H. and Li, M., “Self-assembly of highly oriented lamellar nanoparticle-phospholipid nanocomposites on solid surfaces,” Journal of American Chemical Society, 129, 11332-+(2007).
77. Groves, J. T., Mahal, L. K. and Bertozzi, C. R. Langmuir, “Control of cell adhesion and growth with micropatterned supported lipid membranes,” 17, 5129-5133 (2001).
78. Tang, F. and Hughes, J. A., “Synthesis of a single-tailed cationic lipid and investigation of its transfection,” Journal of Controlled Release, 62, 345-358 (1999).
79. Fayad, W., Rickardson, L., Haglund, C., Olofsson, M. H., D'Arcy, P., Larsson, R., Linder, S. and Fryknas, M., “Identification of agents that induce apoptosis of multicellular tumour spheroids: enrichment for mitotic inhibitors with hydrophobic properties,” Chemical Biology and Drug Design, 78, 547-557 (2011).
80. J.-W. Zhu, H. Nagasawa, F. Nagura, S. B. Mohamad, Y. Uto, K. Ohkura and H. Hori, Bioorg. Med. Chem., 8, 455-463 (2000).

What is claimed is:

1. A product comprising one or more arrays of multilayer structures, wherein the product is formed by a method comprising the following steps:
(a) forming one or more respective openings in a stencil, wherein the one or more respective openings have respective peripheral edges,
(b) disposing the stencil on a surface of a substrate,
(c) filling the one or more respective openings in the stencil on the surface of the substrate with one or more respective dispersions, wherein each of the one or more respective dispersions comprises one or more liquid continuous media and one or more respective dispersed materials, and
(d) evaporating the one or more liquid continuous media from each of the respective dispersions in the one or more respective openings in the stencil on the surface of the substrate to thereby form one or more patterned arrays of the one or more respective dispersed materials along the respective peripheral edges of the one or more respective openings in the stencil on the surface of the substrate.

2. A product of claim 1, wherein the one or more respective dispersed materials comprise one or more liquids.

3. A product of claim 1, wherein the one or more respective dispersed materials comprise one or more gels.

4. A product of claim 1, wherein the one or more respective dispersed materials comprise one or more liquid crystals.

5. A product of claim 1, wherein the one or more respective dispersed materials comprise one or more organic solids.

6. A product of claim 1, wherein the one or more respective dispersed materials comprise one or more metals.

7. A product of claim 1, wherein the one or more respective dispersed materials comprise one or more semiconductor materials.

8. A product of claim 1, wherein the one or more respective dispersed materials comprise one or more polymers.

9. A product of claim 1, wherein the one or more respective dispersed materials comprise one or more glasses.

10. A method comprising the following steps:
(a) forming one or more respective openings in a stencil, wherein the one or more respective openings have respective peripheral edges,
(b) disposing the stencil on a surface of a substrate,
(c) filling the one or more respective openings in the stencil on the surface of the substrate with one or more respective dispersions, wherein each of the one or more respective dispersions comprises one or more liquid continuous media and one or more respective dispersed materials, and
(d) evaporating the one or more liquid continuous media from each of the respective dispersions in the one or more respective openings in the stencil on the surface of the substrate to thereby form one or more patterned arrays of the one or more respective dispersed materials along the respective peripheral edges of the one or more respective openings in the stencil on the surface of the substrate.

11. A product of claim 10, wherein the one or more respective dispersed materials comprise one or more liquids.

12. A product of claim 10, wherein the one or more respective dispersed materials comprise one or more gels.

13. A product of claim 10, wherein the one or more respective dispersed materials comprise one or more liquid crystals.

14. A product of claim 10, wherein the one or more respective dispersed materials comprise one or more organic solids.

15. A product of claim 10, wherein the one or more respective dispersed materials comprise one or more metals.

16. A product of claim 10, wherein the one or more respective dispersed materials comprise one or more semiconductor materials.

17. A product of claim 10, wherein the one or more respective dispersed materials comprise one or more polymers.

18. A product of claim 10, wherein the one or more respective dispersed materials comprise one or more glasses.

19. A product comprising one or more arrays of multilayer structures, wherein the product is formed by a method comprising the following steps:
(a) disposing one or more pairs of barriers on a surface of a substrate to thereby form one or more respective spaces between the one or more pairs of barriers, wherein the one or more pairs of barriers have one or more respective edges,
(b) filling the one or more respective spaces between the one or more pairs of barriers with one or more respective dispersions, wherein each of the respective dispersions comprises one or more liquid continuous media and one or more respective dispersed materials, and
(c) evaporating the one or more liquid continuous media from each of the one or more respective dispersions in the one or more respective spaces between the one or more pairs of barriers to thereby form one or more patterned arrays of the one or more respective dispersed materials along the one or more respective edges of the one or more pairs of barriers.

20. A product of claim 19, wherein the one or more respective dispersed materials comprise one or more liquids.

21. A product of claim 19, wherein the one or more respective dispersed materials comprise one or more gels.

22. A product of claim 19, wherein the one or more respective dispersed materials comprise one or more liquid crystals.

23. A product of claim 19, wherein the one or more respective dispersed materials comprise one or more organic solids.

24. A product of claim 19, wherein the one or more respective dispersed materials comprise one or more metals.

25. A product of claim 19, wherein the one or more respective dispersed materials comprise one or more semiconductor materials.

26. A product of claim 19, wherein the one or more respective dispersed materials comprise one or more polymers.

27. A product of claim 19, wherein the one or more respective dispersed materials comprise one or more glasses.

28. A method comprising the following steps:
(a) disposing one or more pairs of barriers on a surface of a substrate to thereby form one or more respective spaces between the one or more pairs of barriers, wherein the one or more pairs of barriers have one or more respective edges,
(b) filling the one or more respective spaces between the one or more pairs of barriers with one or more respective dispersions, wherein each of the respective dispersions comprises one or more liquid continuous media and one or more respective dispersed materials, and
(c) evaporating the one or more liquid continuous media from each of the one or more respective dispersions in the one or more respective spaces between the one or more pairs of barriers to thereby form one or more patterned arrays of the one or more respective dispersed materials along the one or more respective edges of the one or more pairs of barriers.

29. A product of claim 28, wherein the one or more respective dispersed materials comprise one or more liquids.

30. A product of claim 28, wherein the one or more respective dispersed materials comprise one or more gels.

31. A product of claim 28, wherein the one or more respective dispersed materials comprise one or more liquid crystals.

32. A product of claim 28, wherein the one or more respective dispersed materials comprise one or more organic solids.

33. A product of claim 28, wherein the one or more respective dispersed materials comprise one or more metals.

34. A product of claim 28, wherein the one or more respective dispersed materials comprise one or more semiconductor materials.

35. A product of claim 28, wherein the one or more respective dispersed materials comprise one or more polymers.

36. A product of claim 28, wherein the one or more respective dispersed materials comprise one or more glasses.
